(19) United States
(12) Patent Application Publication
(10) Pub. No.: US 2013/0178377 A1
(43) Pub. Date: Jul. 11, 2013

(54) METAPROTEOMIC METHOD FOR DIAGNOSIS OF BACTERIURIA, UROGENITAL TRACT AND KIDNEY INFECTIONS FROM URINARY PELLET SAMPLES

(71) Applicant: J. Craig Venter Institute, Rockville, MD (US)

(72) Inventors: Rembert Pieper, Washington, DC (US); Moojin Suh, Clarksburg, MD (US); Derrick Fouts, Gaithersburg, MD (US); Karen Nelson, Sandy Spring, MD (US)

(73) Assignee: J. Craig Venter Institute, Rockville, MD (US)

(21) Appl. No.: 13/728,106

(22) Filed: Dec. 27, 2012

Related U.S. Application Data
(60) Provisional application No. 61/585,421, filed on Jan. 11, 2012.

(51) Int. Cl. G06F 19/18; C12Q 1/04

(52) U.S. Cl. CPC G06F 19/18; C12Q 1/04
USPC 506/8

(57) ABSTRACT

Described herein are highly accurate metaproteomic based methods for diagnosing urogenital and kidney infections, which are easy to perform and that also provide information regarding the extent of the infection, the causative agent(s) and the nature of the host response.

### RELATED APPLICATIONS

This application is a non-provisional patent application which claims priority to U.S. Provisional Application No. 61/585,421 filed Jan. 11, 2012. The entire contents of this application is hereby incorporated by reference.

### BACKGROUND

Urinary tract infection (UTI) is among the most common conditions that lead to hospital visits, and catheter-associated UTI (CAUTI) is the most frequent health care-associated infection in the United States (Saint et al. Annals of Internal Medicine 150:877-884 (2009)). In fact, about one-half of all people will contract a UTI at some point during their lifetimes (Schmiemann et al., Deutsches Ärzteblatt International 107:361-367 (2010)). UTI can have serious complications, particularly in children, people with diabetes, the elderly and people with compromised immune systems (Foxman B., Am J Med 113:5-13 (2002); Juthani-Mehta et al., J Am Geriatr Soc 55:1072-1077 (2007)).

Frequently, UTI is diagnosed based on relatively unspecific patient symptoms and a few clinical criteria alone, a process that can have an error rate of as high as 33% (Schmiemann et al., Deutsches Ärzteblatt International 107:361-367 (2010)). In other cases, diagnosis is done by microbiologic culture, which, despite being considered the diagnostic “gold standard,” is slow, labor intensive and often subject to false-negative or false-positive results (Wang et al., American Journal of Clinical Pathology 133:577-582 (2010)). The deficiencies of current diagnostic methods can lead to misdiagnosis and ineffective treatments (the infection-causing agents are not identified) or unnecessary patient treatments (colonization with a microbial agent does not lead to any disease symptoms). These deficiencies can result in the spread of antibiotic-resistant microbes and suboptimal patient outcomes, an especially serious problem in the hospital environment where UTI accounts for 40% of all the acquired infections (Chenoweth and Saint, Infectious Disease Clinics of North America 25:103-115 (2011)). A particular problem in the hospital environment are CAUTIs, which most frequently are associated with the insertion of indwelling urinary catheters in patients for a time period of several days or longer to facilitate bladder voiding when the urethra is obstructed. CAUTIs lead to substantial morbidity and mortality, and the incidence of bacteriuria in catheterized patients varies between 3% and 10% per day (Haley et al., American Journal of Medicine 70:947-959 (1981)). CAUTI is frequently associated with bacterial biofilms forming on the luminal or outer surface of the catheter, and such biofilms are recalcitrant towards antibiotic treatment.

Current diagnostic methods do not reveal much information regarding the nature of the pathogen(s) colonizing the subject's urogenital tract and/or kidney (if there is, in fact, such colonization present). An additional weakness of current diagnostic methods is that they are generally uninformative with regard to the status of the subject's (mammalian host's) antimicrobial and immune responses to the urogenital tract and/or kidney infectious agent. For example, currently used diagnostic methods may not reveal situations where antibiotic administration is unnecessary because the infectious agent does not cause harm or the subject's immune response is successfully fighting off the infectious agent on its own. Lack of symptoms in the context of bacterial colonization of the urogenital tract is referred to as asymptomatic bacteriuria (Chenoweth and Saint, Infectious Disease Clinics of North America 25:103-115 (2011)). Current diagnostic methods are not effective in discerning asymptomatic bacteriuria from UTI.

The most frequently used method to identify urogenital tract and/or kidney infectious agents is urine culture. Urine cultures reveal information on the colonizing microbes that grow under the selected in vitro growth conditions and are easily identifiable by use of microscopic and microbiological staining methods. In a urine culture, bacteria favoring aerobic growth conditions grow faster than bacteria preferring microaerophilic-to-anaerobic growth conditions. Bacteria derived from a CAUTI biofilm may also grow less rapidly in a urine culture because the same silicone/latex surface environment of a catheter is not present. In summary, a urine culture provides little information on relative abundances of microbes in a urine sample and may fail to identify the majority of microbial agents actually present.

Clinical chemistry methods used to diagnose UTI are not very specific, quantitatively not very accurate and do not identify the microbial pathogen(s) causing the UTI. For example, the nitrite concentration assay detects elevated levels of nitrite, a product of anaerobic respiration of bacteria in the urogenital tract, but does not identify the bacteria producing the nitrite. Determining a patient's white blood cell counts can provide an approximate measure of urothelial infiltration with leukocytes, which are eventually released into the urinary tract lumen. However, white blood cell counts do not identify the microbial pathogen(s) and only assess on a very superficial level whether an immune response is activated in the urogenital tract. Finally, the leukocyte esterase assay, which measures the combined esterase enzyme activities in all leukocyte populations, neither identifies the microbial pathogen(s) nor does it determine the cellular origin of the enzyme and natural substrate specificity. The enzyme may also be partially inactivated following release into the urine. In addition, both the nitrite and leukocyte esterase assays are prone to quantitative errors because of chemical compounds and pH conditions present in urine that perturb measurement accuracy.

Therefore, there is great need for improved, more accurate and specific methods for the diagnosis of urogenital tract and kidney infections including those related to CAUTIs, including culture-free microbial identification and more comprehensive molecular assessments of the status of the host organisms' antimicrobial and immune responses.

### SUMMARY

Methods described herein provide: (1) a culture-free method for the identification of microbial colonization of the urogenital tract; if the microbes are bacteria, this represents bacteriuria; (2) a method for the identification of human host proteins released from the urothelial cells, bladder cells and infiltrating immune cells; these proteins are physically associated with the bacteria in the urine or form separate insoluble aggregates precipitating upon centrifugation at 1,500 to 5,000×g; (3) a method to distinguish asymptomatic bacteriuria from urinary tract infection; (4) a culture-free method for the identification of bacterial species associated with a biofilm on the urothelial surface, the external indwelling catheter surface or the internal indwelling catheter surface; (5) a method for the assessment of the mammalian (e.g., human) inflammatory response to microbial colonization of the urogenital tract; (6) a method for the assessment of the mammalian (e.g., human) anti-microbial response to colonization of the urogenital tract; and (7) a method for the identification of uncultivable bacteria colonizing the human urogenital tract (e.g., bacteria that do not grow under standard culture conditions used in the urological clinic).

At the core of the methodologies described herein is shotgun proteomic analysis of urinary pellets. In this shotgun approach, proteins are identified and may be quantified in a highly parallel fashion by mass spectrometry (MS). Prior to analysis, urinary pellet proteins may be cleaved into peptide fragments. In addition, one or more consecutive liquid chromatography (LC) separation steps may be performed to decrease peptide complexity in the sample prior to MS analysis—a process referred to as LC-MS/MS from here on. MS/MS refers to the tandem mass spectrometry mode where the information content for peptide identification is derived from the peptide ion mass-to-charge ratio (m/z) (MS¹ analysis mode) and subsequently generated m/z values of fragment ions with amino acid sequence information (MS² analysis mode).

To identify all proteins of origin in an automated fashion, LC-MS/MS requires a subsequent computational database search step that compares experimental mass spectra (MS¹ and MS² data) with theoretical mass spectra for peptides represented in a database. The term metaproteomics is defined herein as proteomic analysis of a mixture of species and searching the MS data with a compilation of protein sequence databases that represent at least some of the species in the mixture. The mixture may contain more than microbial species colonizing a mammalian host organism, for example, it may include host proteins.

In general, the methods include the steps of: (a) preparing a urinary pellet from a patient sample; (b) generating a complex protein mixture from the urinary pellet; and (c) performing a metaproteomic analysis on the mixture. The metaproteomic analysis may identify proteins of urogenital tract-colonizing microbes. It may also identify proteins released by the mammalian host into the urine.

A urinary pellet may be prepared from a patient sample by centrifuging the sample and re-suspending it in a buffered solution.

A complex protein mixture may be prepared from the urinary pellet by subjecting the urinary pellet to conditions such that the potentially present microbial and host organism cells are lysed and proteins solubilized to form a protein mixture

Protein digestion may be performed on the protein mixture prior to analysis, for example using an enzyme such as trypsin or other endoprotease (e.g., LysN, LysC or GluC).

Metaproteomic analysis of a protein mixture, which is prepared from a urinary pellet may be performed using LC-MS or LC-MS/MS to generate mass spectral data. The LC-MS or LC-MS/MS data can be processed to yield protein identifications based on statistically significant peptide-spectral matches (PSMs). The relative quantity of a protein may be estimated from the sum of all statistically significant PSMs matching to the protein. A computational algorithm that computes PSMs, for example the Mascot v2.3 (Matrix Bioscience) or a non-redundant protein sequence database such as the human protein sequence database subset UniRef90 (www.uniprot.org) may be used to perform the analysis, as described in the following examples. Generally, the more PSMs that are detected for a given protein and the smaller the protein's size, the higher the copy number of the protein in the sample.

Microbial and mammalian host proteins may be quantified simultaneously, allowing one to discern between asymptomatic bacteriuria and UTI in a single “one pot” experiment. For example, the relative quantities of host response proteins (as defined herein) may be quantitated in a sample obtained from a subject. If the subject has asymptomatic bacteriuria, these proteins will be present in lower quantities than if the subject has a UTI or kidney infection.

In addition to mass spectrometry analysis, 16S rRNA sequencing-based metagenomic analysis of the urinary pellet may be performed to identify bacterial genuses present in the urinary pellet.

The diagnostic methods described herein are easy to perform in a laboratory with LC-MS/MS capabilities. In addition, they provide a more accurate diagnosis than currently used clinical chemistry and microbiology methods to discern asymptomatic bacteriuria from UTI and yield additional information allowing an interpretation of the severity of inflammation and infection when UTI is diagnosed. The diagnostic methods described herein allow identification of bacterial agents that are difficult or impossible to cultivate under aerobic conditions (urine culture). The diagnostic methods described herein characterize antimicrobial and inflammatory responses associated with activation and chemotaxis of neutrophils to the site of colonization of the urogenital tract with bacteria. This site may represent the urothelial cell surface and/or the urothelial wall-exposed surface of a urinary catheter.

Further features and advantages will become apparent from the following Detailed Description and Claims.

### BRIEF DESCRIPTION OF THE DRAWING

FIG. 1 shows a schematic depicting exemplary interactions between colonizing bacteria and the host's innate immune system during a urinary tract infection.

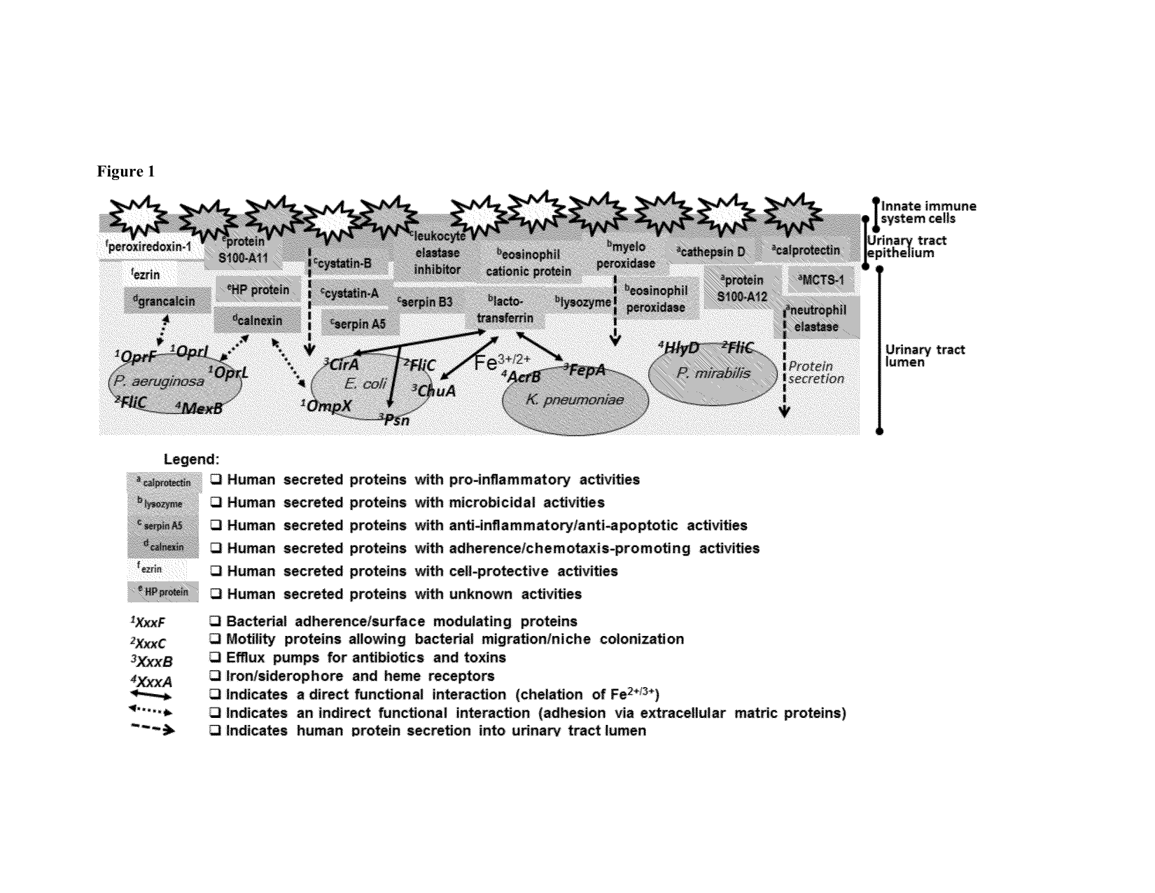

### DETAILED DESCRIPTION

Definitions

As used herein, the following terms and phrases have the meanings described below.

“Diagnosis” and “diagnostic method” refer to any method that provides information regarding the presence, nature and/or cause of an infection in a subject. For example, diagnostic methods can provide information regarding the presence of a urogenital tract and/or kidney infection, the extent of the infection, the identity of an infectious agent colonizing a subject's urogenital tract and/or kidney, and/or the nature of the host response to this colonization.

“Host protein” refers to a protein, which a mammalian subject or host secretes into his or her urine. Host proteins that are useful for diagnosing UTI or kidney infection can include “host response proteins,” for example proteins, which are associated with microbial killing and/or inflammation (e.g. anti-inflammatory, cell adhesion, immune system activating, cytoskeleton associated, protease inhibitory and anti-apoptotic proteins) and proteins that are highly expressed in macrophages and polymorphonuclear neutrophils as well as proteins associated with a release from neutrophil granules or cytoplasms during degranulation and/or release from neutrophils during extracellular trap formation. Exemplary proteins are listed in Table 2A. Host innate immune defense mechanisms reflecting high abundances of proteins listed in Table 2A in the urinary pellet include: (1) opsonization of pathogens and degranulation of secondary granules of polymorphonuclear neutrophils (Weichhart et al., European Journal of Clinical Investigations, 38 (SH2):29-38 (2008)); (2) formation of neutrophil extracellular traps where released secondary granule proteins (myeloperoxidase, neutrophil elastase) initiate cell lysis and release nuclear materials into the urinary tract lumen; the chromatin-containing materials can trap and potentially kill trapped bacteria (von Kloeckertz-Blickwede and Nizet, Journal of Molecular Medicine 87:775-783 (2009)).

Host proteins may also include proteins that are not involved in the host's response, “non-response proteins” such as host proteins, which are generally expressed by the urothelium and released into the urinary tract lumen, independent of the presence of a microbial pathogen, exemplary non-response proteins are listed in Table 2B.

“LC-MS” or “LC-MS/MS” refers to a process in which one or more consecutive liquid chromatography (LC) separation steps is performed to decrease peptide complexity in the sample prior to MS analysis.

“MS/MS” refers to the tandem mass spectrometry mode where the information content for peptide identification is derived from the peptide ion mass-to-charge ratio (m/z) (MS¹ analysis mode) and subsequently generated m/z values of fragment ions with amino acid sequence information (MS² analysis mode).

“Metaproteomic” refers to a proteomic analysis of a mixture of species using an appropriate mass spectrometer (MS) to generate MS data and searching the MS data with a compilation of protein sequence databases that represent at least some of the species in the mixture.

“Microbial proteins associated with urinary tract infections” refer to proteins expressed by urogenital tract colonizing microbes. Certain of these proteins may be involved with microbial survival in the urogenital tract (e.g., iron acquisition proteins, reactive nitrogen and reactive oxygen species, detoxifying enzymes, cell surface proteins, which enable mobility). Examples of microbial proteins that are associated with urinary tract infections are provided in Table 1. Examples of microbial proteins that are associated with urinary tract infections and may contribute to antibiotic resistance and/or tolerance, include: outer membrane porins (OmpA, OmpX, OmpW, and OmpC), subunits of efflux pumps (AcrA, TolC), which may be expressed by many different Gram-negative bacterial pathogens and efflux pumps such as MexA/MexB, which are specific for a urinary tract pathogen (e.g. Pseudomonas aeruginosa).

“m/z value” refers to the mass-to-charge ratio of a peptide which can be determined experimentally in a mass spectrometric measurement and predicted in silico from a database.

“Sample” refers to a urine sample or a preparation made from a urethral catheter-associated biofilm.

“Urogenital tract colonizing microbe” refers to an organism, which may reside in a subject's urogenital tract or kidney. Examples include bacteria, such as Lactobacillus delbrueckii, Lactobacillus jensenii, Lactobacillus gasseri, Corynebacterium urealyticum, uropathogenic Escherichia coli, Peptoniphilus asaccharolyticus, Klebsiella pneumonia, Klebsiella oxytoca, Streptococcus pneumoniae, Prevotella intermedia, Anaerococcus vaginalis, Staphylococcus epidermidis, Proteus mirabilis, Pseudomonas aeruginosa, Finegoldia magna, Enterococcus faecalis, Enterococcus faecium, Morganella morganii, Enterobacter hormaechei or Ureaplasma urealyticum. Schistosoma haematobium is a human parasite, which causes chronic urogenital tract inflammation due to the long-term deposition of eggs in the urothelium and their persistence in this tissue. Hosts harboring this parasite have a high rate of bladder cancer. Exemplary urinary tract or kidney infection-associated fungal pathogens include Candida albicans, Candida glabrata or Candida utilis.

Methods

Metaproteomic methods described herein were used to analyze urinary pellets from individuals who had apparently contracted urinary tract infections. Such urinary pellets contained not only pathogenic bacteria colonizing the urinary tract of the patient, but also host proteins associated with microbial killing and inflammation (host response proteins). The presence of such proteins as a panel can serve as a diagnostic indicator of infection. An important aspect of the invention described herein is that the analysis starts with the isolation of a urinary pellet from a subject followed by metaproteomic analysis of this pellet. Most urine proteomic analysis methods used for clinical purposes pertain to the discovery of disease biomarkers from the soluble phase of the collected urine samples following centrifugation at 1,500 to 5,000×g. The urinary pellet is frequently discarded.

The analyses described herein reveal that a urinary pellet isolated in the context of a UTI is not only enriched in pathogenic and/or non-pathogenic microbial pathogens that colonize the urogenital tract but also in host proteins that are needed for the immune defense against the pathogen and cause local inflammation resulting in urinary tract infection symptoms. Thus featured herein are simultaneous proteomic methods for identifying proteins derived from microbial species and host proteins required for the defense against the colonizing microbial species. The metaproteomic diagnostic methods described herein can be used to rapidly identify both the nature of the infectious agent(s) and the host organism's responses directed towards the infectious agent(s). For example, using the methods described herein, a single experiment may allow identification of many bacterial species based on the identified proteins of a urinary pellet sample. The colonization with the bacteria may result in asymptomatic bacteriuria or symptomatic bacteria (e.g., bacterial colonization is eliciting inflammatory and antimicrobial responses against one or more of the present bacteria). Using the methods described herein, symptomatic bacteriuria (urogenital tract infection) is associated with the identification and high quantities of proteins with antibacterial, pro-inflammatory and pro-apoptotic activities. This subset of proteins is particularly useful for the diagnosis of UTI if, simultaneously, proteins derived from one or several pathogenic microbial agents are identified. This subset of proteins is particularly useful for the diagnosis of UTI if, simultaneously, proteins derived from pathogenic microbial agents with stress response and survival functions are identified. The identification of host response is particularly useful for the diagnosis of UTI. For example, the identification of proteins with antibacterial, pro-inflammatory and pro-apoptotic activities is particularly useful for the diagnosis of UTI, if these proteins are also associated with the release from neutrophil granules, the release from the neutrophil cytoplasm during degranulation and/or the release from neutrophils during extracellular trap formation. Exemplary host response proteins are listed in Table 2A.

Because semi-quantitative protein measurements in shotgun proteomic experiments do not yield absolute quantities, the PSM-based protein quantities of host response proteins should be normalized with PSM-based quantities of proteins generally present in the urothelium, for example non-response proteins that are shed into the urinary tract lumen. Examples of proteins that are generally expressed by the urothelium and released into the urinary tract lumen, independent of the presence of a microbial pathogen, are listed in Table 2B. After normalization of the PSM quantities, an assessment of urinary tract infection can be made. A high ratio of PSM quantities for host response proteins versus PSM quantities for host non-response proteins indicates that the subject has a urinary tract or kidney infection if a microbial pathogen is also identified. A low ratio of PSM quantities for host response proteins versus PSM quantities for non-response proteins indicates absence of a UTI or kidney infection.

Infiltration with neutrophils and other phagocytic cells as well as their activation (degranulation, extracellular neutrophil traps), observed at the level of proteins that characterize this activation is associated with urothelial tissue damage. Local inflammation and urothelial tissue damage can result in the UTI symptoms. The methods described herein enable the diagnosis of UTIs, even if the patient symptoms are vague (occult UTI) or the subject lacks symptoms although bacteria have been identified as colonizing the urogenital tract (asymptomatic bacteriuria).

Methods described herein provide: (1) a culture-free method for the identification of microbial colonization of the urogenital tract; if the microbes are bacteria, this represents bacteriuria; (2) a method for the identification of human host proteins released from the urothelial cells, bladder cells and infiltrating immune cells; these proteins are physically associated with the bacteria in the urine or form separate insoluble aggregates precipitating upon centrifugation at 1,500 to 5,000×g; (3) a method to distinguish asymptomatic bacteriuria from urinary tract infection; (4) a culture-free method for the identification of bacterial species associated with a biofilm on the urothelial surface, the external indwelling catheter surface or the internal indwelling catheter surface; (5) a method for the assessment of the mammalian (e.g., human) inflammatory response to microbial colonization of the urogenital tract; (6) a method for the assessment of the mammalian (e.g., human) anti-microbial response to colonization of the urogenital tract; and (7) a method for the identification of uncultivable bacteria colonizing the human urogenital tract (e.g., bacteria that do not grow under standard culture conditions used in the urological clinic).

The methods described herein are useful for the identification of a urogenital tract and/or kidney infection-associated agent colonizing the urogenital tract and/or kidney of a subject. The methods can include the steps of: (a) centrifuging a urine sample of the subject or a urethral catheter-associated surface biofilm sample of the subject to create a urinary pellet; (b) subjecting the urinary pellet to conditions such that bacteria in the urinary pellet, if present, are lysed and proteins in the urinary pellet are solubilized to form a protein mixture; (c) performing a mass spectrometry-based shotgun proteomics analysis on the protein mixture to generate mass spectral data; and (d) identifying proteins from the urinary pellet by comparing the mass spectral data generated in step (c) with theoretical mass spectra generated from one or more databases that collectively include genome-derived protein sequences from a plurality of urinary tract or kidney infection-associated infectious agents. In general, the presence of at least one protein (e.g., at least 2, 3, 4, 5, 6, 7 or 8 proteins) from a urogenital tract or kidney infection-associated infectious agent in the urinary pellet indicates the colonization with that infectious agent in the urogenital tract and/or kidney of the subject.

Table 1 provides a list of bacterial proteins frequently observed when urinary tract infection with a bacterial pathogen is diagnosed. Many of these bacterial proteins are expressed to adapt to and survive in the urinary tract environment. Some of these proteins, such as iron-acquisition and flagellar proteins, have also been designated virulence-associated factors.

TABLE 1
List of bacterial proteins frequently identified in cases of urinary tract infections

| Protein | Stress re. | E. coli | P. mira | P. aeru | E. horm | K. pneu | E. faec |
|---|---|---|---|---|---|---|---|
| outer membrane receptor (YiuR) | Iron | | x | | | | |
| alkyl hydroperoxide reductase subunit C (AhpC) | ROS | x | x | x | X | x | X |
| heme/hemoglobin transport protein (ChuS) | Iron | x | | | | | |
| thiol peroxidase (Tpx) | ROS | x | x | x | x | x | X |
| pesticin receptor (FyuA) | Iron | x | | | | | |
| SitA metal ion-binding protein (SitA) | Iron | x | | | | | |
| nitric oxide dioxygenase (HmpA) | RNS | x | | | | x | |
| osmotically induced periplasmic protein (OsmY) | OSM | x | | | x | x | |
| cold shock-like protein CspC (CspC) | HS/CS | x | | | x | x | |
| molybdate transporter peripl. protein (ModA) | | x | | | x | x | |
| salicylate synthase Irp9 (Irp9) | iron | x | | | | | |
| glycoprotein/polysaccharide metabolism protein | | | | | x | | |
| (YbaY) | | | | | | | |
| superoxide dismutase, Mn (SodA) | ROS | x | x | X | x | x | X |
| outer membr. ferrienterobactin receptor (FepA) | iron | x | | | x | x | |
| colicin I receptor (CirA) | iron | x | | | | x | |
| thioredoxin (TrxA) | ROS | | | | | | |
| outer membrane protein X (OmpX) | VF | x | | | x | x | |
| alkyl hydroperoxide reductase subunit F (AhpF) | ROS | x | x | x | x | x | X |
| peptidoglycan associated lipoprotein (OprL for P. aeru; | | x | | x | x | x | |
| Pal for other bacteria) | | | | | | | |
| peroxidase/catalase HPI (KatG) | ROS | x | x | x | x | x | X |
| superoxide dismutase, Fe (Sodb) | ROS | x | | | | x | |
| N-acetylneuraminate lyase (NanA) | | x | | | | | |
| cold shock-like protein CspE (CspE) | HS/CS | x | | | | x | |
| iron ABC transporter periplasmic iron-binding | iron | | | | | x | |
| protein (AfeA) | | | | | | | |
| AhpC/TSA family antioxidant protein | ROS | | | | | x | |
| endocarditis specific antigen (PsaA) | | | | | | | X |
| ferrous iron transport protein B (FeoA/FeoB) | iron | | | | | | X |
| NADH peroxidase (Npr) | ROS | | | | | | X |
| thioredoxin disulfide reductase (TrxB) | ROS | x | x | x | x | x | X |
| cold acclimation protein B (CapB) | HS/CS | | | x | | | |
| outer membrane protein A (OmpA) | | x | x | x | x | x | |
| Flagellar protein, type A/B (FliC) | VF | x | x | x | x | | |

Legend, Table 1:
Stress re: stress response;
ROS: reactive oxygen species;
RNS: reactive nitrogen species;
HS/CS: heat shock, cold shock;
iron: iron starvation;
OSM: osmotic stress;
VF: virulence factor;
E. coli: Escherichia coli;
P. mira: Proteus mirabilis;
P. aeru: Pseudomonas aeruginosa;
E. horm: Enterobacter hormachei;
K. pneu: Klebsiella pneumoniae;
E. faec: Enterococcus faecalis.

At least one of the peptides identified for a given protein needs to be unique to a microbial species to confidently identify this microbial species. Uropathogenic E. coli has been reported to account for 80% of all UTIs (Anderson et al., Journal of Clinical Microbiology 42:753-758(2004)). The five other bacterial species listed in Table 1 are likely associated with most of the remaining urinary tract infections.

Methods described herein may be useful for determining whether a subject has a urogenital tract or kidney infection caused by colonization with an infectious agent and a host response. The method can include the steps of: (a) centrifuging a urine sample of the subject or a urethral catheter-associated surface biofilm sample of the subject to create a urinary pellet; (b) subjecting the urinary pellet to conditions such that bacteria in the urinary pellet, if present, are lysed and proteins in the urinary pellet are solubilized to form a protein mixture; (c) performing a mass spectrometry-based shotgun proteomics analysis on the solubilized proteins to generate mass spectral data; and (d) identifying proteins from this mixture using spectral data and proteomics-specific algorithms that identify at high confidence peptide-spectral matches (PSMs) computationally. The latter process requires a comparison of experimental mass spectral data generated in step (c) with theoretical mass spectra derived in silico from a host organism (e.g., human) that includes all protein sequences from the genome of the host organism. An example is the non-redundant human protein sequence database subset of UniRef90, www.uniprot.org).

All identified and quantified host organism proteins may provide information on the status of the antimicrobial and immune responses following colonization with one or more microbes which may be identified simultaneously in the metaproteomic analysis. However, the methods described herein provide specific information on host organism proteins associated with antimicrobial and innate immune responses that are launched by the host organism in defense to the colonizing/invading pathogen(s). The proteins that are indicative of such host responses may be released by phagocytic cells and, specifically, neutrophils.

The methods described herein enable the diagnosis of UTIs based on the relative abundance of proteins released by neutrophils compared to the abundance of proteins generally abundant in and shed from urothelial cells during the voiding of urine. The higher the relative abundance of such neutrophil-specific released proteins compared to that of proteins generally associated with presence in the urothelium, the more evident is a host organism response associated with inflammation justifying the diagnosis of UTI. Proteins, which are generally observed in the urothelium and shed into the urine serve the purpose of quantitative data normalization. At least the following twelve host response proteins are likely to be observed as a consequence of a UTI or kidney infection: myeloperoxidase, lactotransferrin, defensin Al, lipocalin, azurocidin, proactivator peptide, cathepsin G, lysozyme, neutrophil elastase, myeloblastin, protein S100-A8 and protein S100-A9. A more extensive protein list is provided in Table 2A.

At least the following twelve host non-response proteins may be identified frequently in a urinary pellet with or without the presence of an infectious agent and/or any indication of inflammation: annexin A1, annexin A2, glutathione S-transferase (P), 14-3-3 zeta/delta protein, serpin A5, serpin B3, cystatin A, cystatin B, cornulin, epidermal fatty-acid binding protein, heat shock protein beta-1 and apolipoprotein D. A more extensive protein list is provided in Table 2B.

TABLE 2A
Table 2A. List of host response proteins

| | | | Function/extracellular |
|---|---|---|---|
| Protein name | Protein annotation | Localization | release |
| lactotransferrin | TRFL_HUMAN | Present in neutrophil | extracellular release |
| | | secretory granules and | |
| | | epithelia of mucosal | |
| | | surfaces | |
| myeloperoxidase | PERM_HUMAN | Present in neutrophil | extracellular release |
| | | secretory granules | |
| neutrophil defensin 1 | DEF1_HUMAN | Present in azurophilic | extracellular release |
| | | neutrophil secretory | |
| | | granules and epithelia of | |
| | | mucosal surfaces | |
| neutrophil elastase | ELNE_HUMAN | Present in azurophilic | extracellular release |
| | | neutrophil secretory | |
| | | granules and cytoplasm | |
| cathepsin G | CATG_HUMAN | Present in neutrophil | extracellular release |
| | | secretory granules | |
| lysozyme C | LYSC_HUMA | Present in neutrophil | extracellular release |
| | | secretory granules and | |
| | | epithelia of mucosal | |
| | | surfaces | |
| neutrophil gelatinase- | NGAL_HUMAN | Present in neutrophil | extracellular release |
| associated lipocalin | | secretory granules | |
| phospholipase B-like 1 | PLBL1_HUMAN | Secreted by neutrophils | extracellular release |
| protein | | and monocytes | |
| cathelicidin antimicrobial | CAMP_HUMAN | Present in secretory | extracellular release |
| peptide | | granules | |
| Azurocidin | CAP7_HUMAN | Present in azurophilic | extracellular release |
| | | neutrophil granules | |
| carcinoembryonic antigen- | CEAM8_HUMAN | | cell surface membrane- |
| related cell adhesion | | | anchored (leukocytes) |
| molecule 8 | | | |
| neutrophil defensin 4 | DEF4_HUMAN | | extracellular release |
| Grancalcin | GRAN_HUMAN | Present in neutrophil | extracellular release and |
| | | secretory granule | granule surface-associated |
| | | membranes | |
| myeloblastin | PRTN3_HUMAN | Present in azurophilic | extracellular release |
| | | neutrophil granules | |
| protein S100-A9 | S10A9_HUMAN | Induces degranulation of | neutrophil cytoplasm and |
| | | neutrophils by a MAPK-dep. | extracellular release via cell |
| | | mechanism; present in | death |
| | | neutrophil cytoplasm | |
| protein S100-A8 | S10A8_HUMAN | Induces degranulation of | neutrophil cytoplasm and |
| | | neutrophils by a MAPK-dep. | extracellular release via cell |
| | | mechanism; present in | death |
| | | neutrophil cytoplasm | |
| integrin alpha-M | ITAM_HUMAN | Neutrophil adherence | cell surface membrane- |
| | | receptor, leukocyte | anchored (leukocytes) |
| | | migration | |
| proactivator polypeptide | SAP_HUMAN | Platelet degranulation/ | lysosomal and extracellular |
| | | activation | release |
| olfactomedin-4 | OLFM4_HUMAN | Present in neutrophils of | extracellular release |
| | | prostate and intestine | |
| neutrophil collagenase | MMP8_HUMAN | Present in neutrophil | extracellular release |
| | | secretory granules | |
| annexin A3 | ANXA3_HUMAN | Involved in neutrophil | neutrophil granule |
| | | degranulation | membrane-anchored |
| prolifin-1 | PROF1_HUMAN | Platelet degranulation/ | extracellular release |
| | | activation | |
| plastin 2 | PLSL_HUMAN | Ubiquitous, neutrophil | actin cytoskeleton |
| | | extracellular traps | |

TABLE 2B
Table 2B. List of host non-response proteins.

| Protein | Protein annotation | Localization | Function |
|---|---|---|---|
| protein S100-P | S100P_HUMAN | | Cell migration/differentiation |
| protein S100-A11 | S10AB_HUMAN | | Cell migration/differentiation |
| annexin A2 | ANXA2_HUMAN | Ubiquitous | Phospholipase inhibitor |
| annexin A1 | ANXA1_HUMAN | Ubiquitous | Anti-apoptosis |
| glutathione S-transferase P | GSTP1_HUMAN | | Anti-apoptosis |
| heat shock protein beta-1 | HSPB1_HUMAN | Ubiquitous | Anti-apoptosis, actin cytoskeleton |
| cystatin B | CYTB_HUMAN | Ubiquitous | Protease inhibitor |
| serpin B3 | SPB3_HUMAN | Squamous epithelium | Protease inhibitor, cell differentiation |
| serpin A5 | IPSP_HUMAN | Ubiquitous | Protease inhibitor |
| epidermal fatty-acid | FABP5_HUMAN | Keratinocytes | Cell migration/differentiation |
| binding protein | | | |
| alpha-actinin 1 | ACTN1_HUMAN | Ubiquitous | Actin cytoskeleton |
| Apolipoprotein D | APOD_HUMAN | Ubiquitous | Negative regulation of inflammation |
| cystatin A | CYTA_HUMAN | | Protease inhibitor |
| 14-3-3 zeta/delta protein | 1433Z_HUMAN | | Anti-apoptosis |
| cornulin | CRNN_HUMAN | Squamous epithelium | Cell adhesion |
| peptidyl-prolyl cis-trans | PPIA_HUMAN | Ubiquitous | Protein folding |
| isomerase A | | | |
| glyceraldehyde-3- | G3P_HUMAN | Ubiquitous | Energy metabolism |
| phosphate dehydrogenase | | | |

Legend, Table 2A/2B:
Localization refers to a high expression level of a protein in a specific cell type or tissue; the term ‘ubiquitous’ refers to proteins not associated with expression a specific cell type or tissue.
Function refers to a major functional role of a protein; it does not exclude other functional roles not listed for a particular protein in Table 2B.
Extracellular release: this column indicates which proteins are released into the extracellular environment by leukocytes, most often from the neutrophil cytoplasm or its secondary granules. Release of the proteins is associated with inflammation and antimicrobial defense.

For use herein, a urinary pellet can be from any mammalian subject, including both human and non-human subjects. The subject may have or may be suspected as having a urogenital tract and/or kidney infection. For example, a subject may be “suspected of having a urogenital tract or kidney infection” if that subject exhibits one or more symptoms of a urogenital tract or kidney infection. Such symptoms are known in the art, and may include painful urination, frequent urination, abdominal pain, cloudy urine, foul smelling urine, fever, accelerated heart rate and/or tenderness at the costovertebral angle. A subject subject may also be “suspected of having a urogenital tract or kidney infection” if he or she is predisposed to having a urogenital tract or kidney infection. Factors that indicate a predisposition for a urogenital tract or kidney infection are known in the art and can include recent urinary catheterization, sexual activity, a family history of urogenital tract infections, and/or diabetes. In general, women are more susceptible to urogenital tract and kidney infections then men.

The methods described herein may include the step of obtaining a urinary pellet prepared from a patient sample (e.g., a urine sample or a urethral catheter-associated biofilm sample). A urinary pellet may be prepared using any method known in the art. For example, a subjects' urine (e.g., 10 to 500 mL of urine) may be subjected to centrifugation at 5,000×g for 15 min at 4° C. to generate a urinary pellet. The pellet may then be isolated from the supernatant by, for example, aspirating or decanting the supernatant from the reaction vessel containing the pellet. The pellet fraction may then be washed with a wash buffer (e.g., a 10-fold volume of PBS). Once prepared, a urinary pellet may be analyzed immediately or may be frozen (e.g., at −80 ° C.) until further analysis.

A urinary pellet may then be prepared into a solubilized protein mixture. Such a protein mixture may be generated using any method available in the art. For example, the urinary pellet may be subjected to conditions such that bacteria in the urinary pellet, if present, are lysed and proteins in the urinary pellet are solubilized to form a protein mixture. Such conditions are known in the art. For example, the urinary pellet may be treated with a detergent (e.g., Triton X-100) and and/or an EDTA solution, followed by sonication, in order to lyse bacteria and solubilize proteins. Exemplary sample preparation methods are provided in Wisniewski et al., Nat Methods 6:356-362 (2009) and Fouts et al., J. Hepatology 56:1283-92 (2012), which are incorporated by reference in its entirety.

A mass spectrometer may be used to analyze the protein mixture. For example, the protein mixture may be directly analyzed using a mass spectrometry-based approach, such as liquid chromatography tandem mass-spectrometry (LC-MS/MS) or liquid chromatography mass-spectrometry (LC-MS). Methods for identifying microorganism using mass spectrometry are described, for example, in Demirev et al., Anal Chem 71:2732-2738 (1999) and Eschelbach et al., Anal Chem 78:1697-1706 (2006), each of which is incorporated by reference in its entirety.

A mass-spectrometry-based shotgun proteomics analysis may be performed on peptides generated from a protein mixture. Such protein-derived peptides can be generated by any appropriate method known in the art. For example, the peptides may be generated according to the methods described in Wisniewski et al., Nat Methods 6:356-362 (2009), which is incorporated by reference in its entirety. Enzymatic digests may be performed on a protein mixture to generate protein-derived peptides, which may then be analyzed by LC-MS and/or LC-MS/MS.

Proteins present in a protein mixture may be analyzed by a shotgun proteomics approach using LC-MS/MS. The shotgun proteomics analysis may comprise a filter-aided tryptic digestion of total protein and application of the protein digest to LC-MS/MS analysis. For example, the tryptic-digested peptides may be subjected to a C₁₈LC-MS/MS analysis on an electrospray ionization tandem mass spectrometer with up-front peptide separation at acidic pH. Methods of proteomic analysis using LC-MS/MS are provided, for example, in Wolters et al., Anal Chem 73:5683-5690 (2001); Peng et al., J Proteome Res 2:43-50 (2003); Kuntumalla et al., BMC Microbiol 11:147 (2011); and Pieper et al., PLoS One 6:26554 (2011), each of which is incorporated by reference in its entirety.

The mass spectral data produced may be interpreted using a metaproteomic approach in order to identify proteins present in the urinary pellet. In general, proteins present in the urinary pellet may be identified by comparing the mass spectral data generated with theoretical mass spectral data generated from one or more databases that collectively include genome-derived protein sequences from a plurality of organisms using one or more databases. Databases of genome-derived protein sequences from various organisms are known in the art and many are publicly available. Exemplary methods of metaproteomic analysis are provided in Verberkmoes et al., ISME J 3:179-189 (2009); and Li et al., PLoS One 6:e26542 (2011), each of which is incorporated by reference in its entirety.

Databases containing genome-derived protein sequences of urogenital tract or kidney infection-associated infectious agents are known in the art and are publically available, for example, from the National Center for Biotechnology Information (NCBI) taxonomy browser at http://www.ncbi.nlm.nih.gov/.

The database(s) may collectively comprise sequences of proteins that confer antibiotic resistance to the urogenital tract or kidney disease-associated infectious agent and the presence of a protein that confers antibiotic resistance indicates that the subject has a urogenital tract or kidney disease caused by colonization with an antibiotic-resistant bacterial pathogen. Proteins that convey antibiotic resistance are known in the art. For example, proteins that convey antibiotic resistance are described in Aminov and Mackie FEMS Microbiol Lett 271:147-161 (2007) and R. Canton Clin Microbial Infect 15 (Suppl. I): 20-25 (2009), each of which is incorporated by reference in its entirety.

The methods described may also include the step of performing 16S rRNA sequencing-based metagenomic analysis of the urinary pellet to identify bacterial genera present in the urinary pellet. In such embodiments, the one or more databases used for mass spectrometry-based shotgun proteomics analysis may collectively include protein sequences of those genera identified by the 16S rRNA sequencing-based metagenomic analysis. The database(s) used for mass spectrometry-based shotgun proteomics analysis may include only protein sequences of those bacterial genera identified by the 16S rRNA sequencing-based metagenomic analysis. The database may include only protein sequences of those genera identified by the 16S rRNA sequencing-based metagenomic analysis and human protein sequences. Methods for performing 16S rRNA sequencing-based metagenomic analysis are known in the art and are described in, for example, Tringe et al., Science 308:554-557 (2005), Eckburg et al., Science 308:1635-1638 (2005) and Manichanh et al., Gut 55:205-211 (2006), each of which is incorporated by reference in its entirety.

The methods described herein may also include the step of performing deep metagenomic sequencing-based analysis of the urinary pellet to identify bacterial species and/or bacterial open reading frames present in the urinary pellet. In deep metagenomic-based sequencing, entire genomes of bacterial organisms present in the urinary pellet are sequenced. In such embodiments, the database(s) may include protein sequences of those bacterial species identified by the deep metagenomic sequencing-based analysis. The database may include only protein sequences of those bacterial species identified by the deep metagenomic sequencing-based analysis. A database(s) may include only protein sequences of those bacterial species identified by the deep metagenomic sequencing-based analysis and human protein sequences. A database(s) may include protein sequences encoded by bacterial open reading frames identified (e.g., sequenced, assembled and/or annotated) in the deep metagenomic sequencing-based analysis. A database(s) may only include protein sequences encoded by bacterial open reading frames identified (e.g., sequenced, assembled and/or annotated) in the deep metagenomic sequencing-based analysis. Methods for performing deep metagenomic sequencing-based analysis are known in the art and are described in, for example, von Mering et al., Science 315:1126-1130 (2007), Grice et al., Genome Res 18:1043-1050 (2008), and Qin et al., Nature 464:59-65 (2010), each of which is incorporated by reference in its entirety.

Mass spectral searches described herein may use both bacterial protein sequence databases and a non-redundant human protein sequence database. As described herein, proteins present in the urinary pellet may be identified through the use of a mass spectrometry algorithm that identifies peptides (and the proteins these peptides are derived from) through a computational matching and statistical analysis process in which experiment and theoretical mass spectra are compared. This approach may determine the taxonomy of bacteria to the species level via protein sequence analysis. Furthermore, since metaproteomic data are semi-quantitative, abundant proteins identified from a sample can be determined from the scores (provided by the mass spectrometric algorithm) and allow interpretation of key biological activities contributed by the urinary tract invading bacteria and the host (e.g. the human host's inflammatory and bactericidal responses) simultaneously. This parallel and semi-quantitative analysis of inflammatory proteins expressed and secreted by the host's immune cells, such as macrophages and neutrophils recruited to the urothelium during an infectious process, can be used as indicators of the infection (rather than simple colonization). Protein biomarkers that are found to be particularly useful diagnostically may alternatively be used in immunoassays or other diagnostic procedures.

All publications, including GI and GenBank Accession numbers mentioned herein are hereby incorporated by reference in their entirety as if each individual publication or patent was specifically and individually indicated to be incorporated by reference. In case of conflict, the present application, including any definitions herein, will control.

The invention, now being generally described, will be more readily understood by reference to the following example, which is included merely for purposes of illustration of certain aspects and embodiments of the present invention, and is not intended to limit the invention. The following example describes experimental methods, details of the databases searched and detailed results. The results consist of four tables (Table 3, 4, 5, and 6), each of which represents a metaproteomic dataset from a human donor's urinary pellet analyzed by LC-MS/MS.

### EXAMPLE

### Detailed Metaproteomic Method for Diagnosing Bacteriuria, Urogenital Tract and Kidney Infections from Urinary Pellet Samples.

Methods and Materials. Approximately 50 ml of urine was collected from human subjects. The urine was stored at 4° C. for up to 6 hours and centrifuged for 15 min at 5,000×g at 4° C. The pellet was recovered, retaining ˜1 ml of residual urine supernatant to avoid disturbing the urinary pellet. Addition of ˜10 ml ice-cold phosphate-buffered saline PBS was followed by gentle shaking of the tube and centrifugation for 15 min at 5,000×g at 4° C. The wet urinary pellet was frozen at −80° C. until analyzed.

To lyse cells in the urinary pellet and solubilize the contents, 2 ml of 10 mM ammonium bicarbonate buffer containing 0.1% Triton-X100, 0.5% octylglucoside, 5 μg/ml leupeptin, 10 mM EDTA and 2 mM BAM was added to the pellet. The pellets were heated to 85° C. for 5 min and sonicated at the amplitude 4 (Misonex 3000 sonicator) in 30 s on/15 s off cycles 10 times in an ice bath. The suspension was centrifuged for 15 min at 16,100×g at 4° C. and the supernatant was recovered. Following an estimation of the protein contents using Coomassie Blue-stained SDS-PAGE analysis, up to 20 ug solubilized urinary pellet protein was applied to a Microcon filter device (MW cutoff 10,000), trypsin was added at a 1:50 ratio followed by application of the Filter-Aided Sample Preparation protocol, as described in Allegrucci et al., J Bacteriol 188:2325-35 (2006), which is incorporated by reference in its entirety.

The protein digestion mixture recovered from the filtrate of FASP processing was lyophilized and reconstituted in 50 μl 0.1% formic acid. Twenty μl of the sample was subjected to reversed phase C₁₈ LC-MS/MS analysis on an Agilent 1200 solvent delivery system coupled to the nano-electrospray ionization source of an LTQ-XL ion trap mass spectrometer Thermo Electron LLC). The peptide separation was performed on a BioBasic C₁₈ column (75 μm×10 cm; New Objective, Woburn, Mass.). The LC-MS/MS instrument workflow, the experimental and data analysis parameters were previously described in Pieper et al., PLoS One 6:e26554 (2011), which is incorporated by reference in its entirety.

The instrument was calibrated prior at the beginning of each day LC-MS/MS experiments were performed with 200 nmol human [Glu¹]-fibrinopeptide B (M.W. 1570.57), verifying that elution times with a CH₃CN gradient varied less than 10% and that peaks representing ion counts had widths at half-height of <0.25 min, signal/noise ratios >200 and peak heights >10⁷. Following quality control and calibration of the LTQ-XL mass spectrometer, loading a 20 μl urinary precipitate lysate sample was followed by trapping and wash (salt removal) of the peptide mixture on a C₁₈ trapping cartridge at a flow rate of 0.01 ml/min for 3 min. Peptides were eluted from the C₁₈ cartridge and separated on the C₁₈ column with 122 min binary gradient runs from 97% solvent A (0.1% formic acid) to 80% solvent B (0.1% formic acid, 90% AcCN) at a flow rate of 350 nl/min.

Spectra were acquired in automated MS/MS mode, with the top five parent ions selected for fragmentation in scans of the m/z range 350-2,000 and with a dynamic exclusion setting of 90 sec, deselecting repeatedly observed ions for MS/MS. All peptide fractions from a given urinary precipitate lysate sample were run consecutively on the LC-MS/MS system. The LTQ search parameters (+1 to +3 ions) included mass error tolerances of ±1.4 Da for peptide precursor ions and ±0.5 Da for peptide fragment ions. The search engine used for peptide identifications was Mascot v.2.3 (Matrix Science). Search parameters allowed one missed tryptic cleavage, and were set for oxidation of methionine residues as a variable modification. The customized protein sequence database is comprised of individual genome-wide protein sequence databases for the following species (and strains):
1) Lactobacillus delbrueckii subsp. bulgaricus PB2003/044-T3-4 AEAT01000000 (http://www.ncbi.nlm.nih.gov/Taxonomy/Browser/wwwtax.cgi?id=784613)
2) Lactobacillus jensenii JV-V16 ACGQ02000000 (http://www.ncbi.nlm.nih.gov/Taxonomy/Browser/wwwtax.cgi?id=525329)
3) Lactobacillus gasseri JV-V03 ACG002000000 (http://www.ncbi.nlm.nih.gov/Taxonomy/Browser/wwwtax.cgi?id=525326)
4) Corynebacterium urealyticum (already exist in Mascot—C_urealytic_DSM7109: c_urealyticum_DSM7109_{—}20110713.fasta) http://www.ncbi.nlm.nih.gov/Taxonomy/Browser/wwwtax.cgi?mode=Info&id=43771&1v1 =3&p=mapview&p=has_linkout&p=blasturl&p=genome_blast&lin=f&keep=1&srchmod e=1&unlock
5) Escherichia coli UPEC (already exist in Mascot—E_{—}coli_UPEC_CFT073: uropathogenic_{—}e_{—}coli_CFT073_{—}20110630.fasta) http://www.ncbi.nlm.nih.gov/Taxonomy/Browser/wwwtax.cgi?mode=Info&id=199310&1v1=3&p=mapview&p=has_linkout&p=blasturl&p=genome_blast&lin=f&keep=1&srchmod e=1&unlock
6) Peptoniphilus asaccharolyticus (http://www.ncbi.nlm.nih.gov/Taxonomy/Browser/wwwtax.cgi?mode=Info&id=1258&1v1=3&lin=f&keep=1&srchmode=1 &unlock)
7) Klebsiella pneumoniae (already exist in Mascot—K_{—}pneumoniae_{—}342: k_{—}pneumoniae_{—}342_{—}20110630.fasta) http://www.ncbi.nlm.nih.gov/Taxonomy/Browser/wwwtax.cgi?mode=Info&id=507522&1v1=3&lin=f&keep=1&srchmode=1&unlock
8) Streptococcus pneumoniae (http://www.ncbi.nlm.nih.gov/Taxonomy/Browser/wwwtax.cgi?mode=Info&id=1313&1v1=3&lin=f&keep=1&srchmode=1&unlock)
9) Prevotella intermedia (http://www.ncbi.nlm.nih.gov/Taxonomy/Browser/wwwtax.cgi?mode=Info&id=28131&1v1=3&lin=f&keep=1&srchmode=1&unlock)
10) Anaerococcus vaginalis (http://www.ncbi.nlm.nih.gov/Taxonomy/Browser/wwwtax.cgi?mode=Info&id=33037&1v1=3&lin=f&keep=1&srchmode=1&unlock)
11) Staphylococcus epidermidis (http://www.ncbi.nlm.nih.gov/Taxonomy/Browser/wwwtax.cgi?mode=Info&id=1282&1v1=3&lin=f&keep=1&srchmode=1&unlock)
12) Proteus mirabilis (already exist in Mascot—P_{—}mirabilis_HI4320: p_mirabilis_HI4320_{—}20110630.fasta) http://www.ncbi.nlm.nih.gov/Taxonomy/Browser/wwwtax.cgi?mode=Info&id=529507&1v1=3&lin=f&keep=1&srchmode=1&unlock
13) Pseudomonas aeruginosa (already exist in Mascot—P_{—}aeruginosa_PAO1: p_{—}aeruginosa_PAO1_{—}20110630.fasta) http://www.ncbi.nlm.nih.gov/Taxonomy/Browser/wwwtax.cgi?mode=Info&id=208964&1v1=3&lin=f&keep=1&srchmode=1&unlock
14) Finegoldia magna (http://www.ncbi.nlm.nih.gov/Taxonomy/Browser/wwwtax.cgi?mode=Info&id=1260&1v1=3&lin=f&keep=1&srchmode=1&unlock)
15) Enterococcus faecalis (http://www.ncbi.nlm.nih.gov/Taxonomy/Browser/wwwtax.cgi?mode=Info&id=1351&1v1=3&lin=f&keep=1&srchmode=1&unlock)
16) Enterococcus faecium (http://www.ncbi.nlm.nih.gov/Taxonomy/Browser/wwwtax.cgi?mode=Info&id=1352&1v1=3&lin=f&keep=1&srchmode=1&unlock)
17) Morganella morganii (already exist in Mascot—M_{—}morganii: m_{—}morganii_{—}20110630.fasta) http://www.ncbi.nlm.nih.gov/Taxonomy/Browser/wwwtax.cgi?mode=Info&id=582&1v1=3&lin=f&keep=1&srchmode=1&unlock
18) Enterobacter hormaechei (http://www.ncbi.nlm.nih.gov/Taxonomy/Browser/wwwtax.cgi?mode=Info&id=158836&1v1=3&lin=f&keep=1&srchmode=1&unlock)
19) Ureaplasma urealyticum (http://www.ncbi.nlm.nih.gov/Taxonomy/Browser/wwwtax.cgi?mode=Info&id=2130&1v1=3&lin=f&keep=1&srchmode=1&unlock)
20) Human database is Uniref90: downloaded on 20110817 http://www.ebi.ac.uk/uniprot/database/download.html, filter on organism: Homo sapiens

Mascot search peptide false discovery rates (FDR) were determined by searching an in silico randomized protein sequence dataset from genome-based databases mentioned above and set at 1%. Furthermore, stringent criteria for peptide-spectral matches (q-value<=0.01; PEP-value<=10-4) were set using the Mascot Percolator algorithm. This algorithm improves discrimination between correct and incorrect PSMs, particularly when the database sequence space is large (www.matrixscience.com/help/percolator_help.html) (Fouts et al., Journal of Translational Medicine 10:174-186 (2012)).

Results.

The results of the metaproteomic analysis of human subject 1 is provided in Table 3.

TABLE 3

| Accession Number | Score | Mass | PSMs | Description | Notes |
|---|---|---|---|---|---|
| UniRef90_P02788 | 1485 | 80014 | 25 (17) | Lactotransferrin n = 24 Tax = Hominoidea | NIP |
| | | | | RepID = TRFL_HUMAN | |
| UniRef90_E9PFJ3 | 854 | 65573 | 17 (11) | uromodulin n = 3 Tax = Simiiformes | USP* |
| | | | | RepID = E9PFJ3_HUMAN | |
| UniRef90_P06702 | 683 | 13291 | 10 (6) | Protein S100-A9 n = 5 Tax = Hominoidea | NIP |
| | | | | RepID = S10A9_HUMAN | |
| UniRef90_P05164 | 628 | 84784 | 19 (11) | Myeloperoxidase n = 6 Tax = Catarrhini | NIP |
| | | | | RepID = PERM_HUMAN | |
| UniRef90_P12429 | 472 | 36524 | 11 (9) | Annexin A3 n = 15 Tax = Eutheria RepID = ANXA3_HUMAN | NIP |
| UniRef90_P04083 | 364 | 38918 | 9 (5) | Annexin A1 n = 14 Tax = Eutheria RepID = ANXA1_HUMAN | USP |
| UniRef90_P61626 | 345 | 16982 | 9 (4) | Lysozyme C n = 9 Tax = Hominoidea RepID = LYSC_HUMAN | NIP |
| UniRef90_P05109 | 308 | 10885 | 7 (4) | Protein S100-A8 n = 5 Tax = Hominoidea | NIP |
| | | | | RepID = S10A8_HUMAN | |
| UniRef90_P35579 | 240 | 227646 | 3 (1) | Myosin-9 n = 67 Tax = Tetrapoda RepID = MYH9_HUMAN | USP |
| UniRef90_B4DRW1 | 230 | 52069 | 7 (5) | cDNA FLJ55805, highly similar to Keratin, type II | |
| | | | | cytoskeletal 4 n = 2 Tax = Hominidae | |
| | | | | RepID = B4DRW1_HUMAN | |
| UniRef90_B4DR52 | 207 | 18087 | 3 (2) | Histone H2B n = 5 Tax = Euarchontoglires | |
| | | | | RepID = B4DR52_HUMAN | |
| UniRef90_P08311 | 201 | 29161 | 11 (5) | Cathepsin G n = 3 Tax = Homininae RepID = CATG_HUMAN | NIP |
| UniRef90_P19012 | 196 | 49409 | 6 (5) | Keratin, type I cytoskeletal 15 n = 6 Tax = Hominoidea | |
| | | | | RepID = K1C15_HUMAN | |
| gi\|197284727 | 177 | 74047 | 2 (2) | outer membrane receptor [Proteus mirabilis HI4320] | BACT |
| gi\|206576207 | 175 | 18908 | 2 (2) | peptidoglycan-associated lipoprotein [Klebsiella | BACT |
| | | | | pneumoniae 342] | |
| UniRef90_B4DWC9 | 160 | 32176 | 1 (1) | Cathepsin S RepID = B4DWC9_HUMAN | |
| gi\|197285073 | 139 | 20798 | 4 (3) | alkyl hydroperoxide reductase subunit C [Proteus | BACT |
| | | | | mirabilis HI4320] | |
| UniRef90_B2MUD5 | 101 | 21048 | 4 (2) | Neutrophil elastase (Fragment) n = 1 Tax = Homo sapiens | NIP |
| | | | | RepID = B2MUD5_HUMAN | |
| UniRef90_P04264 | 92 | 66170 | 7 (4) | Keratin, type II cytoskeletal 1 n = 7 Tax = Eutheria | |
| | | | | RepID = K2C1_HUMAN | |
| UniRef90_P41439 | 87 | 28532 | 1 (1) | Folate receptor gamma n = 8 Tax = Catarrhini | |
| | | | | RepID = FOLR3_HUMAN | |
| UniRef90_P80188 | 83 | 22745 | 1 (1) | Neutrophil gelatinase-associated lipocalin n = 7 | NIP |
| | | | | Tax = Hominidae RepID = NGAL_HUMAN | |
| UniRef90_P59665 | 64 | 10536 | 4 (2) | Neutrophil defensin 1 n = 8 Tax = Homininae | NIP |
| | | | | RepID = DEF1_HUMAN | |
| UniRef90_UPI0001BEF2DB | 58 | 23163 | 2 (1) | Fab 537-10D, light chain n = 1 Tax = Homo sapiens | |
| | | | | RepID = UPI0001BEF2DB | |
| gi\|197284658 | 47 | 40796 | 5 (2) | outer membrane porin [Proteus mirabilis HI4320] | BACT |
| UniRef90_D6RBE9 | 44 | 24739 | 3 (1) | Annexin 5 n = 3 Tax = Eutheria RepID = D6RBE9_HUMAN | USP |
| UniRef90_P35527 | 43 | 62255 | 5 (2) | Keratin, type I cytoskeletal 9 n = 4 Tax = Catarrhini | |
| | | | | RepID = K1C9_HUMAN | |
| UniRef90_P07602 | 37 | 59899 | 2 (2) | Proactivator polypeptide n = 27 Tax = Simiiformes | NIP |
| | | | | RepID = SAP_HUMAN | |
| UniRef90_P07737 | 35 | 15216 | 2 (1) | Profilin-1 n = 21 Tax = Theria RepID = PROF1_HUMAN | NIP |
| UniRef90_P20160 | 31 | 27325 | 4 (3) | Azurocidin n = 2 Tax = Homininae RepID = CAP7_HUMAN | NIP |
| UniRef90_Q9HDC9 | 30 | 46622 | 2 (1) | Adipocyte plasma membrane-associated protein n = 12 | |
| | | | | Tax = Eutheria RepID = APMAP_HUMAN | |
| UniRef90_P11215 | 23 | 128410 | 2 (1) | Integrin alpha-M n = 6 Tax = Simiiformes | NIP |
| | | | | RepID = ITAM_HUMAN | |
| UniRef90_P28676 | 20 | 24223 | 1 (1) | Grancalcin n = 9 Tax = Catarrhini RepID = GRAN_HUMAN | NIP |
| gi\|197283924 | 17 | 69298 | 1 (1) | chaperone protein DnaK [Proteus mirabilis HI4320] | BACT |
| UniRef90_F5H0N0 | 17 | 37725 | 3 (1) | Uncharacterized protein n = 11 Tax = Simiiformes | |
| | | | | RepID = F5H0N0_HUMAN | |
| UniRef90_P49913 | 17 | 19517 | 2 (1) | Cathelicidin antimicrobial peptide n = 7 Tax = Hominoidea | NIP |
| | | | | RepID = CAMP_HUMAN | |
| UniRef90_P31997 | 16 | 38415 | 2 (1) | Carcinoembryonic antigen-related cell adhesion | NIP |
| | | | | molecule 8 n = 3 Tax = Homininae RepID = CEAM8_HUMAN | |
| UniRef90_P06703 | 15 | 10230 | 1 (1) | Protein S100-A6 n = 12 Tax = Eutheria | |
| | | | | RepID = S10A6_HUMAN | |

Legend.
Accession numbers are from the Uniprot or the NCBI Entrez Med databases (see method section).
The scores are derived from the database searches with the Mascot v.2.3 algorithms (Matrix Bioscience). A high score is indicative of more peptide identifications for a given protein combined with higher confidence identifications of the peptides.
Masses are the relative molecular mass values for the entire protein, as annotated in the searched databases.
PSMs (peptide-spectral matches) provide the semi-quantitative abundance value for an identified protein. Only statistically significant peptide-spectral matches for a protein sequence are counted. In parentheses are the rank = 1 peptides. The latter peptide counts (rank = 1) are selected for semi-quantitative analysis.
Description lists the protein name, protein name abbreviation and species (e.g. a bacterial species and human).
Notes are provided for the association of proteins with expression in as well as secretion by neutrophils and inflammation (NIP), abundance in and secretion or shedding by urothelium (USP), and a bacterial pathogen (BACT).
USP* Uromodulin is released into the urine in very high abundance. There is no evidence that this protein is released in higher abundance when bacteria colonize the urogenital tract and inflammation occurs.
Result interpretation. Metaproteomic data indicate that the urinary pellet obtained from this human subject contain proteins derived from two different bacterial species, each of which is known to be able to cause urinary tract infections (Proteus mirabilis, Klebsiella pneumoniae). The relatively high abundance and the large number of proteins that are associated with neutrophils and particularly release from neutrophils during neutrophil degranulation and neutrophil extracellular trap formation (NIPs) compared to the abundance of proteins generally associated with the urothelium (USPs) indicate that there is substantial activation of neutrophils and neutrophil-induced inflammation, resulting in urinary tract infection.

The results of the metaproteomic analysis of human subject 2 is provided in Table 4.

TABLE 4

| Accession Number | Score | Mass | PSMs | Description | Notes |
|---|---|---|---|---|---|
| UniRef90_E9PFJ3 | 4187 | 65573 | 294 (149) | uromodulin n = 3 Tax = Simiiformes RepID = E9PFJ3_HUMAN | USP* |
| UniRef90_P13646 | 3564 | 49900 | 155 (77) | Keratin, type I cytoskeletal 13 n = 13 Tax = Simiiformes | |
| | | | | RepID = K1C13_HUMAN | |
| UniRef90_P04264 | 1367 | 66170 | 68 (35) | Keratin, type II cytoskeletal 1 n = 7 Tax = Eutheria | |
| | | | | RepID = K2C1_HUMAN | |
| gi\|334125226 | 869 | 95925 | 16 (12) | chaperone protein ClpB [Enterobacter hormaechei ATCC | BACT |
| | | | | 49162] | |
| gi\|206576484 | 829 | 39657 | 24 (14) | outer membrane protein C [Klebsiella pneumoniae 342] | BACT |
| gi\|206577432 | 740 | 85444 | 34 (17) | formate acetyltransferase [Klebsiella pneumoniae 342] | BACT |
| gi\|206580041 | 717 | 69164 | 26 (14) | chaperone protein DnaK [Klebsiella pneumoniae 342] | BACT |
| gi\|334122237 | 716 | 51388 | 12 (8) | pyruvate kinase [Enterobacter hormaechei ATCC 49162] | BACT |
| gi\|334123809 | 522 | 57209 | 30 (12) | chaperone GroEL [Enterobacter hormaechei ATCC | BACT |
| | | | | 49162] | |
| gi\|334123375 | 498 | 30468 | 23 (13) | elongation factor EF1B [Enterobacter hormaechei ATCC | BACT |
| | | | | 49162] | |
| UniRef90_P06702 | 478 | 13291 | 24 (12) | Protein S100-A9 n = 5 Tax = Hominoidea | NIP |
| | | | | RepID = S10A9_HUMAN | |
| gi\|206577622 | 462 | 28301 | 24 (9) | 2,3-bisphosphoglycerate-dependent phosphoglycerate | BACT |
| | | | | mutase [Klebsiella pneumoniae 342] | |
| gi\|206577987 | 448 | 18250 | 10 (8) | glucose-specific phosphotransferase enzyme IIA | BACT |
| | | | | component [Klebsiella pneumoniae 342] | |
| UniRef90_P05164 | 445 | 84784 | 20 (9) | Myeloperoxidase n = 6 Tax = Catarrhini | NIP |
| | | | | RepID = PERM_HUMAN | |
| gi\|334121697 | 432 | 78683 | 8 (6) | oligopeptidase A [Enterobacter hormaechei ATCC 49162] | BACT |
| gi\|206580108 | 418 | 41038 | 5 (4) | hypothetical protein KPK_3511 [Klebsiella pneumoniae | BACT |
| | | | | 342] | |
| gi\|334122926 | 386 | 22488 | 11 (9) | alkyl hydroperoxide reductase C [Enterobacter | BACT |
| | | | | hormaechei ATCC 49162] | |
| gi\|334123379 | 385 | 30046 | 4 (4) | 2,3,4,5-tetrahydropyridine-2,6-dicarboxylate N- | BACT |
| | | | | succinyltransferase [Enterobacter hormaechei ATCC | |
| | | | | 49162] | |
| gi\|334123165 | 383 | 87769 | 13 (5) | ATP-dependent protease La [Enterobacter hormaechei | BACT |
| | | | | ATCC 49162] | |
| gi\|206577508 | 381 | 37528 | 6 (5) | outer membrane protein A [Klebsiella pneumoniae 342] | BACT |
| gi\|197284609 | 379 | 61379 | 8 (6) | 30S ribosomal protein S1 [Proteus mirabilis HI4320] | BACT |
| gi\|206576266 | 367 | 19924 | 3 (3) | flavodoxin [Klebsiella pneumoniae 342] | BACT |
| gi\|206580165 | 352 | 61316 | 15 (8) | glucose-6-phosphate isomerase [Klebsiella pneumoniae | BACT |
| | | | | 342] | |
| gi\|206578717 | 340 | 39434 | 13 (9) | fructose-bisphosphate aldolase [Klebsiella pneumoniae | BACT |
| | | | | 342] | |
| gi\|206581095 | 340 | 66661 | 4 (3) | aspartyl-tRNA synthetase [Klebsiella pneumoniae 342] | BACT |
| gi\|161486272 | 333 | 18742 | 4 (4) | DNA starvation/stationary phase protection protein Dps | BACT |
| | | | | [Escherichia coli CFT073] | |
| gi\|206577583\| | 319 | 11957 | 10 (7) | thioredoxin [Klebsiella pneumoniae 342] | BACT |
| UniRef90_P02788 | 298 | 80014 | 14 (8) | Lactotransferrin n = 24 Tax = Hominoidea | NIP |
| | | | | RepID = TRFL_HUMAN | |
| gi\|334125094 | 297 | 52177 | 5 (3) | inosine-5′-monophosphate dehydrogenase | BACT |
| | | | | [Enterobacter hormaechei ATCC 49162] | |
| gi\|206579534 | 296 | 71035 | 7 (7) | chaperone protein HtpG [Klebsiella pneumoniae 342] | BACT |
| gi\|206578835 | 270 | 18528 | 3 (3) | outer membrane protein X [Klebsiella pneumoniae 342] | BACT |
| gi\|206577768 | 258 | 65779 | 6 (4) | dihydrolipoyllysine-residue acetyltransferase [Klebsiella | BACT |
| | | | | pneumoniae 342] | |
| gi\|161486316 | 249 | 63620 | 6 (4) | prolyl-tRNA synthetase [Escherichia coli CFT073] | BACT |
| UniRef90_P05109 | 248 | 10885 | 24 (6) | Protein S100-A8 n = 5 Tax = Hominoidea | NIP |
| | | | | RepID = S10A8_HUMAN | |
| UniRef90_P04083 | 240 | 38918 | 10 (6) | Annexin A1 n = 14 Tax = Eutheria RepID = ANXA1_HUMAN | USP |
| gi\|26250521 | 239 | 49705 | 5 (4) | transcription termination factor Rho [Escherichia coli | BACT |
| | | | | CFT073] | |
| gi\|206576288 | 239 | 67818 | 16 (2) | PTS system mannitol-specific EIICBA component | BACT |
| | | | | [Klebsiella pneumoniae 342] | |
| gi\|161486328 | 215 | 50942 | 2 (2) | dihydrolipoamide dehydrogenase [Escherichia coli | BACT |
| | | | | CFT073] | |
| UniRef90_D6PXK4 | 215 | 80206 | 4 (2) | Alpha actinin 4 short isoform n = 8 Tax = Eutheria | NIP |
| | | | | RepID = D6PXK4_HUMAN | |
| UniRef90_P01876 | 199 | 38486 | 6 (3) | Ig alpha-1 chain C region n = 2 Tax = Homo sapiens | |
| | | | | RepID = IGHA1_HUMAN | |
| gi\|206581027 | 195 | 32024 | 5 (4) | isochorismatase [Klebsiella pneumoniae 342] | BACT |
| UniRef90_B4DQ53 | 192 | 15688 | 2 (2) | Uncharacterized protein n = 1 Tax = Homo sapiens | |
| | | | | RepID = B4DQ53_HUMAN | |
| gi\|26247927 | 192 | 8375 | 3 (3) | major outer membrane lipoprotein [Escherichia coli | BACT |
| | | | | CFT073] | |
| gi\|26249980 | 187 | 24596 | 3 (2) | ribulose-phosphate 3-epimerase [Escherichia coli | BACT |
| | | | | CFT073] | |
| gi\|206576099 | 186 | 52585 | 5 (2) | aminoacyl-histidine dipeptidase [Klebsiella pneumoniae | BACT |
| | | | | 342] | |
| gi\|206577099 | 177 | 40924 | 9 (4) | putrescine ABC transporter, periplasmic putrescine- | BACT |
| | | | | binding protein [Klebsiella pneumoniae 342] | |
| gi\|206575799 | 173 | 22233 | 3 (3) | FKBP-type 22 kDa peptidyl-prolyl cis-trans isomerase FklB | BACT |
| | | | | [Klebsiella pneumoniae 342] | |
| UniRef90_P62979 | 170 | 18296 | 4 (2) | Ubiquitin-40S ribosomal protein S27a n = 48 | |
| | | | | Tax = Coelomata RepID = RS27A_HUMAN | |
| gi\|26250238 | 166 | 68123 | 16 (3) | PTS system, mannitol-specific IIABC component | BACT |
| | | | | [Escherichia coli CFT073] | |
| UniRef90_P80188 | 163 | 22745 | 5 (3) | Neutrophil gelatinase-associated lipocalin n = 7 | NIP |
| | | | | Tax = Hominidae RepID = NGAL_HUMAN | |
| gi\|334125780 | 161 | 97597 | 4 (2) | translation initiation factor IF-2 [Enterobacter | BACT |
| | | | | hormaechei ATCC 49162] | |
| UniRef90_P07355 | 159 | 38808 | 4 (4) | Annexin A2 n = 28 Tax = Eutheria RepID = ANXA2_HUMAN | USP |
| gi\|334122685 | 159 | 26969 | 4 (2) | arginine ABC superfamily ATP binding cassette | BACT |
| | | | | transporter, binding protein [Enterobacter hormaechei | |
| | | | | ATCC 49162] | |
| gi\|206578928 | 157 | 23442 | 1 (1) | fructose-6-phosphate aldolase 2 [Klebsiella pneumoniae | BACT |
| | | | | 342] | |
| gi\|26248549 | 156 | 33962 | 2 (2) | 1-phosphofructokinase [Escherichia coli CFT073] | BACT |
| gi\|334126120 | 155 | 27239 | 11 (6) | uridine phosphorylase [Enterobacter hormaechei ATCC | BACT |
| | | | | 49162] | |
| UniRef90_P59665 | 154 | 10536 | 6 (4) | Neutrophil defensin 1 n = 8 Tax = Homininae | NIP |
| | | | | RepID = DEF1_HUMAN | |
| gi\|334122958 | 150 | 82734 | 3 (2) | ferrienterobactin receptor [Enterobacter hormaechei | BACT |
| | | | | ATCC 49162] | |
| gi\|206576051 | 147 | 12542 | 3 (3) | ribosomal subunit interface protein [Klebsiella | BACT |
| | | | | pneumoniae 342] | |
| gi\|206576724 | 138 | 27801 | 2 (1) | amino acid ABC transporter, periplasmic amino acid- | BACT |
| | | | | binding protein [Klebsiella pneumoniae 342] | |
| gi\|206579100 | 133 | 27149 | 5 (2) | glutamine ABC transporter, periplasmic glutamine- | BACT |
| | | | | binding protein [Klebsiella pneumoniae 342] | |
| gi\|26246587 | 132 | 57737 | 9 (4) | Alkyl hydroperoxide reductase subunit F [Escherichia coli | BACT |
| | | | | CFT073] | |
| UniRef90_B4DR52 | 131 | 18087 | 5 (2) | Histone H2B n = 5 Tax = Euarchontoglires | NIP |
| | | | | RepID = B4DR52_HUMAN | |
| gi\|334124969 | 129 | 28285 | 3 (1) | lysine/arginine/ornithine ABC superfamily ATP binding | BACT |
| | | | | cassette transporter, binding protein [Enterobacter | |
| | | | | hormaechei ATCC 49162] | |
| gi\|206579376 | 129 | 41237 | 7 (2) | phosphoglycerate kinase [Klebsiella pneumoniae 342] | BACT |
| UniRef90_Q6UX06 | 123 | 57529 | 3 (2) | Olfactomedin-4 n = 7 Tax = Catarrhini | NIP |
| | | | | RepID = OLFM4_HUMAN | |
| UniRef90_P31949 | 122 | 11847 | 3 (2) | Protein S100-A11 n = 16 Tax = Simiiformes | USP |
| | | | | RepID = S10AB_HUMAN | |
| gi\|206577548 | 121 | 77680 | 8 (3) | translation elongation factor G [Klebsiella pneumoniae | BACT |
| | | | | 342] | |
| gi\|26249087 | 112 | 58745 | 2 (1) | glutamate-cysteine ligase [Escherichia coli CFT073] | BACT |
| UniRef90_Q6N094 | 112 | 53264 | 6 (2) | Putative uncharacterized protein DKFZp686O01196 n = 3 | |
| | | | | Tax = Homo sapiens RepID = Q6N094_HUMAN | |
| gi\|334124536 | 108 | 51651 | 7 (1) | pyruvate kinase [Enterobacter hormaechei ATCC 49162] | BACT |
| gi\|334124376 | 105 | 15618 | 3 (1) | DNA-binding protein VicH [Enterobacter hormaechei | BACT |
| | | | | ATCC 49162] | |
| gi\|26248090 | 102 | 30070 | 2 (2) | transcriptional regulator kdgR [Escherichia coli CFT073] | BACT |
| UniRef90_B1ALW1 | 101 | 9674 | 2 (2) | Thioredoxin n = 3 Tax = Catarrhini RepID = B1ALW1_HUMAN | |

Legend.
Accession numbers are from the Uniprot or the NCBI Entrez Med databases (see method section).
The scores are derived from the database searches with the Mascot v.2.3 algorithms (Matrix Bioscience). A high score is indicative of more peptide identifications for a given protein combined with higher confidence identifications of the peptides.
Masses are the relative molecular mass values for the entire protein, as annotated in the searched databases.
PSMs (peptide-spectral matches) provide the semi-quantitative abundance value for an identified protein. Only statistically significant peptide-spectral matches for a protein sequence are counted. In parentheses are the rank = 1 peptides. The latter peptide counts (rank = 1) are selected for semi-quantitative analysis.
Description lists the protein name, protein name abbreviation and species (e.g. a bacterial species and human).
Notes are provided for the association of proteins with expression in as well as secretion by neutrophils and inflammation (NIP), abundance in and secretion or shedding by urothelium (USP), and a bacterial pathogen (BACT).
USP* Uromodulin is released into the urine in very high abundance. There is no evidence that this protein is released in higher abundance when bacteria colonize the urogenital tract and inflammation occurs.
Result interpretation. Metaproteomic data indicate that the urinary pellet obtained from this human subject contain proteins derived from three different bacterial species, each of which is known to be able to cause urinary tract infections (Escherichia coli, Klebsiella pneumonia, Enterobacter hormachei). The bacterial proteins are highly prevalent indicative of substantial bacterial colonization. The relatively high abundance of proteins that are associated with neutrophils and particularly release from neutrophils during neutrophil extracellular trap formation (NIPs) compared to the abundance of proteins generally associated with the urothelium (USPs) indicate that there is activation of neutrophils and neutrophil-induced inflammation, resulting in urinary tract infection.

The results of the metaproteomic analysis of human subject 3 is provided in Table 5.

TABLE 5

| Accession Number | Score | Mass | PSMs | Description | Notes |
|---|---|---|---|---|---|
| UniRef90_E9PFJ3 | 2494 | 65573 | 135 (67) | Uromodulin n = 3 Tax = Simiiformes RepID = E9PFJ3_HUMAN | USP* |
| gi\|334123809 | 1407 | 57209 | 34 (22) | chaperone GroEL [Enterobacter hormaechei ATCC 49162] | BACT |
| UniRef90_P13646 | 1167 | 49900 | 45 (25) | Keratin, type I cytoskeletal 13 n = 13 Tax = Simiiformes | |
| | | | | RepID = K1C13_HUMAN | |
| UniRef90_P04264 | 1038 | 66170 | 27 (20) | Keratin, type II cytoskeletal 1 n = 7 Tax = Eutheria | |
| | | | | RepID = K2C1_HUMAN | |
| gi\|206576484 | 1026 | 39657 | 22 (16) | outer membrane protein C [Klebsiella pneumoniae 342] | BACT |
| gi\|206579376 | 1008 | 41237 | 31 (20) | phosphoglycerate kinase [Klebsiella pneumoniae 342] | BACT |
| gi\|206577548 | 966 | 77680 | 28 (13) | translation elongation factor G [Klebsiella pneumoniae 342] | BACT |
| gi\|334122618 | 932 | 61289 | 26 (16) | 30S ribosomal protein S1 [Enterobacter hormaechei ATCC | BACT |
| | | | | 49162] | |
| gi\|206578717 | 775 | 39434 | 16 (10) | fructose-bisphosphate aldolase [Klebsiella pneumoniae | BACT |
| | | | | 342] | |
| UniRef90_P06702 | 730 | 13291 | 31 (10) | Protein S100-A9 n = 5 Tax = Hominoidea | NIP |
| | | | | RepID = S10A9_HUMAN | |
| UniRef90_P04083 | 492 | 38918 | 14 (7) | Annexin A1 n = 14 Tax = Eutheria RepID = ANXA1_HUMAN | USP |
| gi\|206580165 | 414 | 61316 | 10 (3) | glucose-6-phosphate isomerase [Klebsiella pneumoniae | BACT |
| | | | | 342] | |
| gi\|206576507 | 404 | 76965 | 8 (4) | oligopeptidase A [Klebsiella pneumoniae 342] | BACT |
| gi\|206578835 | 391 | 18528 | 5 (3) | outer membrane protein X [Klebsiella pneumoniae 342] | BACT |
| gi\|334122625 | 377 | 85296 | 11 (5) | formate acetyltransferase [Enterobacter hormaechei ATCC | BACT |
| | | | | 49162] | |
| gi\|206579040 | 363 | 76869 | 12 (3) | polyribonucleotide nucleotidyltransferase [Klebsiella | BACT |
| | | | | pneumoniae 342] | |
| gi\|206577768 | 359 | 65779 | 8 (4) | dihydrolipoyllysine-residue acetyltransferase [Klebsiella | BACT |
| | | | | pneumoniae 342] | |
| gi\|206580041 | 357 | 69164 | 13 (4) | chaperone protein DnaK [Klebsiella pneumoniae 342] | BACT |
| gi\|206581102 | 347 | 29401 | 6 (3) | FKBP-type peptidyl-prolyl cis-trans isomerase FkpA | BACT |
| | | | | [Klebsiella pneumoniae 342] | |
| gi\|334122237 | 324 | 51388 | 10 (5) | pyruvate kinase [Enterobacter hormaechei ATCC 49162] | BACT |
| gi\|206576699 | 323 | 98185 | 7 (4) | translation initiation factor IF-2 [Klebsiella pneumoniae | BACT |
| | | | | 342] | |
| gi\|26246978 | 316 | 41143 | 8 (5) | outer membrane protein A [Escherichia coli CFT073] | BACT |
| gi\|26250733 | 304 | 50602 | 3 (2) | argininosuccinate lyase [Escherichia coli CFT073] | BACT |
| gi\|161486316 | 303 | 63620 | 8 (3) | prolyl-tRNA synthetase [Escherichia coli CFT073] | BACT |
| gi\|206579308 | 297 | 31427 | 8 (3) | dihydrodipicolinate synthase [Klebsiella pneumoniae 342] | BACT |
| gi\|206579164 | 295 | 60389 | 7 (5) | dipeptide ABC transporter, periplasmic dipeptide-binding | BACT |
| | | | | protein [Klebsiella pneumoniae 342] | |
| gi\|26250238 | 293 | 68123 | 8 (6) | PTS system, mannitol-specific IIABC component | BACT |
| | | | | [Escherichia coli CFT073] | |
| gi\|206578649 | 290 | 20675 | 3 (2) | ribosome recycling factor [Klebsiella pneumoniae 342] | BACT |
| gi\|206576322 | 285 | 73175 | 6 (5) | colicin I receptor [Klebsiella pneumoniae 342] | BACT |
| UniRef90_Q5RHS7 | 282 | 16027 | 3 (2) | S100 calcium binding protein A2 n = 2 Tax = Catarrhini | |
| | | | | RepID = Q5RHS7_HUMAN | |
| gi\|206577622 | 262 | 28301 | 17 (8) | 2,3-bisphosphoglycerate-dependent phosphoglycerate | BACT |
| | | | | mutase [Klebsiella pneumoniae 342] | |
| gi\|206579548 | 259 | 50948 | 4 (3) | dihydrolipoamide dehydrogenase [Klebsiella pneumoniae | BACT |
| | | | | 342] | |
| gi\|206576547 | 255 | 28661 | 6 (3) | histidine ABC transporter, periplasmic histidine-binding | BACT |
| | | | | protein [Klebsiella pneumoniae 342] | |
| gi\|334123375 | 241 | 30468 | 10 (4) | elongation factor EF1B [Enterobacter hormaechei ATCC | BACT |
| | | | | 49162] | |
| gi\|206576265 | 239 | 12334 | 25 (4) | ribosomal protein L7/L12 [Klebsiella pneumoniae 342] | BACT |
| gi\|334121775 | 232 | 54402 | 5 (3) | ketol-acid reductoisomerase [Enterobacter hormaechei | BACT |
| | | | | ATCC 49162] | |
| gi\|206576051 | 225 | 12542 | 4 (2) | ribosomal subunit interface protein [Klebsiella pneumoniae | BACT |
| | | | | 342] | |
| gi\|26247927 | 223 | 8375 | 10 (3) | major outer membrane lipoprotein [Escherichia coli | BACT |
| | | | | CFT073] | |
| UniRef90_B4DR52 | 222 | 18087 | 4 (2) | Histone H2B n = 5 Tax = Euarchontoglires | |
| | | | | RepID = B4DR52_HUMAN | |
| gi\|334122688 | 219 | 28842 | 5 (3) | arginine ABC superfamily ATP binding cassette transporter, | BACT |
| | | | | binding protein [Enterobacter hormaechei ATCC 49162] | |
| gi\|206578670 | 214 | 13877 | 3 (2) | FeS assembly scaffold SufA [Klebsiella pneumoniae 342] | BACT |
| gi\|206580144 | 212 | 43096 | 4 (3) | maltose ABC transporter, periplasmic maltose-binding | BACT |
| | | | | protein [Klebsiella pneumoniae 342] | |
| gi\|206577987 | 208 | 18250 | 11 (4) | glucose-specific phosphotransferase enzyme IIA | BACT |
| | | | | component [Klebsiella pneumoniae 342] | |
| gi\|334126404 | 207 | 12809 | 4 (2) | cupin domain protein [Enterobacter hormaechei ATCC | BACT |
| | | | | 49162] | |
| gi\|26248869 | 206 | 54949 | 7 (2) | inosine 5′-monophosphate dehydrogenase [Escherichia coli | BACT |
| | | | | CFT073] | |
| gi\|206579534 | 204 | 71035 | 9 (3) | chaperone protein HtpG [Klebsiella pneumoniae 342] | BACT |
| gi\|334123379 | 198 | 30046 | 3 (2) | 2,3,4,5-tetrahydropyridine-2,6-dicarboxylate N- | BACT |
| | | | | succinyltransferase [Enterobacter hormaechei ATCC 49162] | |
| gi\|206578935 | 193 | 39802 | 7 (2) | phosphoserine aminotransferase [Klebsiella pneumoniae | BACT |
| | | | | 342] | |
| gi\|206580252 | 187 | 22386 | 7 (3) | 2-dehydro-3-deoxyphosphogluconate aldolase/4-hydroxy- | BACT |
| | | | | 2-oxoglutarate aldolase [Klebsiella pneumoniae 342] | |
| gi\|161486074 | 185 | 49916 | 6 (4) | xylose isomerase [Escherichia coli CFT073] | BACT |
| gi\|206577295 | 182 | 18697 | 3 (2) | DNA protection during starvation protein [Klebsiella | BACT |
| | | | | pneumoniae 342] | |
| gi\|206578240 | 176 | 39516 | 6 (4) | high-affinity branched-chain amino acid ABC transporter, | BACT |
| | | | | periplasmic leucine-specific-binding protein [Klebsiella | |
| | | | | pneumoniae 342] | |
| gi\|206577583 | 175 | 11957 | 3 (3) | thioredoxin [Klebsiella pneumoniae 342] | BACT |
| gi\|206577933 | 174 | 41756 | 7 (2) | O-succinylhomoserine (thiol)-lyase [Klebsiella pneumoniae | BACT |
| | | | | 342] | |
| gi\|206579921 | 172 | 39412 | 6 (2) | outer membrane porin, OmpF family [Klebsiella | BACT |
| | | | | pneumoniae 342] | |
| gi\|26250771 | 162 | 9529 | 2 (2) | transcriptional regulator HU subunit alpha [Escherichia coli | BACT |
| | | | | CFT073] | |
| gi\|26247234 | 162 | 8634 | 2 (2) | acyl carrier protein [Escherichia coli CFT073] | BACT |
| gi\|26248016 | 160 | 48766 | 7 (2) | glutamate dehydrogenase [Escherichia coli CFT073] | BACT |
| gi\|206579361 | 159 | 15089 | 1 (1) | ribosomal protein S6 [Klebsiella pneumoniae 342] | BACT |
| gi\|206578745 | 157 | 82389 | 3 (3) | ferrienterobactin receptor [Klebsiella pneumoniae 342] | BACT |
| gi\|26248549 | 156 | 33962 | 1 (1) | 1-phosphofructokinase [Escherichia coli CFT073] | BACT |
| gi\|206579637 | 155 | 63499 | 10 (4) | phosphoenolpyruvate-protein phosphotransferase | BACT |
| | | | | [Klebsiella pneumoniae 342] | |
| gi\|206575727 | 155 | 48027 | 11 (6) | trigger factor [Klebsiella pneumoniae 342] | BACT |
| gi\|161486282 | 154 | 19896 | 2 (2) | flavodoxin FldA [Escherichia coli CFT073] | BACT |
| gi\|206579438 | 150 | 15334 | 6 (4) | DNA-binding protein H-NS [Klebsiella pneumoniae 342] | BACT |
| gi\|197286032 | 150 | 71546 | 9 (1) | heat shock protein 90 [Proteus mirabilis HI4320] | BACT |
| gi\|206578029 | 149 | 21178 | 1 (1) | YGGT family protein [Klebsiella pneumoniae 342] | BACT |
| gi\|334125399 | 149 | 45632 | 5 (2) | enolase [Enterobacter hormaechei ATCC 49162] | BACT |
| gi\|206581055 | 148 | 61466 | 2 (1) | oligopeptide ABC transporter, periplasmic oligopeptide- | BACT |
| | | | | binding protein [Klebsiella pneumoniae 342] | |
| gi\|334123373 | 146 | 20754 | 3 (1) | ribosome recycling factor [Enterobacter hormaechei ATCC | BACT |
| | | | | 49162] | |
| UniRef90_P59665 | 139 | 10536 | 7 (4) | Neutrophil defensin 1 n = 8 Tax = Homininae | NIP |
| | | | | RepID = DEF1_HUMAN | |
| gi\|26251040 | 137 | 20635 | 2 (2) | elongation factor P [Escherichia coli CFT073] | BACT |
| gi\|26246955 | 136 | 43826 | 6 (2) | aromatic amino acid aminotransferase [Escherichia coli | BACT |
| | | | | CFT073] | |
| gi\|206580859 | 133 | 15574 | 2 (2) | nucleoside diphosphate kinase [Klebsiella pneumoniae 342] | BACT |
| gi\|26246587 | 130 | 57737 | 10 (3) | Alkyl hydroperoxide reductase subunit F [Escherichia coli | BACT |
| | | | | CFT073] | |
| UniRef90_P05109 | 130 | 10885 | 21 (4) | Protein S100-A8 n = 5 Tax = Hominoidea | NIP |
| | | | | RepID = S10A8_HUMAN | |
| UniRef90_P09211 | 128 | 23569 | 3 (2) | Glutathione S-transferase P n = 4 Tax = Simiiformes | USP |
| | | | | RepID = GSTP1_HUMAN | |
| gi\|26248550 | 122 | 39654 | 3 (1) | bifunctional PTS system fructose-specific transporter | BACT |
| | | | | subunit IIA/HPr protein [Escherichia coli CFT073] | |
| gi\|206577275 | 121 | 56185 | 8 (2) | 2,3-bisphosphoglycerate-independent phosphoglycerate | BACT |
| | | | | mutase [Klebsiella pneumoniae 342] | |
| gi\|206577473 | 121 | 39749 | 3 (2) | 3-isopropylmalate dehydrogenase [Klebsiella pneumoniae | BACT |
| | | | | 342] | |
| gi\|206580108 | 119 | 41038 | 4 (3) | hypothetical protein KPK_3511 [Klebsiella pneumoniae | BACT |
| | | | | 342] | |
| gi\|26250749 | 119 | 43457 | 11 (4) | elongation factor Tu [Escherichia coli CFT073] | BACT |
| gi\|334125823 | 118 | 55270 | 3 (1) | leucyl aminopeptidase [Enterobacter hormaechei ATCC | BACT |
| | | | | 49162] | |
| gi\|206575925 | 113 | 39023 | 2 (1) | high-affinity branched-chain amino acid ABC transporter, | BACT |
| | | | | periplasmic Leu/Ile/Val-binding protein [Klebsiella | |
| | | | | pneumoniae 342] | |
| UniRef90_P06703 | 113 | 10230 | 1 (1) | Protein S100-A6 n = 12 Tax = Eutheria RepID = S10A6_HUMAN | |
| gi\|334122926 | 112 | 22488 | 17 (8) | alkyl hydroperoxide reductase C [Enterobacter hormaechei | BACT |
| | | | | ATCC 49162] | |
| gi\|26248868 | 108 | 59027 | 4 (1) | GMP synthase [Escherichia coli CFT073] | BACT |
| gi\|206578640 | 108 | 32549 | 4 (1) | malate dehydrogenase, NAD-dependent [Klebsiella | BACT |
| | | | | pneumoniae 342] | |
| gi\|334123431 | 106 | 65944 | 7 (1) | pyruvate dehydrogenase complex E2, dihydrolipoamide | BACT |
| | | | | acetyltransferase [Enterobacter hormaechei ATCC 49162] | |
| gi\|26250634 | 106 | 52099 | 4 (2) | glutamine synthetase [Escherichia coli CFT073] | BACT |
| gi\|206578928 | 104 | 23442 | 4 (2) | fructose-6-phosphate aldolase 2 [Klebsiella pneumoniae | BACT |
| | | | | 342] | |
| gi\|206577225 | 103 | 17674 | 2 (2) | thiol peroxidase [Klebsiella pneumoniae 342] | BACT |
| UniRef90_UPI000011049E | 102 | 24407 | 7 (1) | IGG CTM01 FAB (LIGHT CHAIN) n = 2 Tax = Homo sapiens | |
| | | | | RepID = UPI000011049E | |
| gi\|197286616 | 101 | 17697 | 3 (2) | 50S ribosomal protein L10 [Proteus mirabilis HI4320] | BACT |
| gi\|26250199 | 101 | 7399 | 3 (3) | major cold shock protein [Escherichia coli CFT073] | BACT |
| gi\|26248085 | 100 | 7398 | 6 (4) | cold shock-like protein CspC [Escherichia coli CFT073] | BACT |

Legend.
Accession numbers are from the Uniprot or the NCBI Entrez Med databases (see method section).
The scores are derived from the database searches with the Mascot v.2.3 algorithms (Matrix Bioscience). A high score is indicative of more peptide identifications for a given protein combined with higher confidence identifications of the peptides.
Masses are the relative molecular mass values for the entire protein, as annotated in the searched databases.
PSMs (peptide-spectral matches) provide the semi-quantitative abundance value for an identified protein. Only statistically significant peptide-spectral matches for a protein sequence are counted. In parentheses are the rank = 1 peptides. The latter peptide counts (rank = 1) are selected for semi-quantitative analysis.
Description lists the protein name, protein name abbreviation and species (e.g. a bacterial species and human).
Notes are provided for the association of proteins with expression in as well as secretion by neutrophils and inflammation (NIP), abundance in and secretion or shedding by urothelium (USP), and a bacterial pathogen (BACT).
USP* Uromodulin is released into the urine in very high abundance. There is no evidence that this protein is released in higher abundance when bacteria colonize the urogenital tract and inflammation occurs.
Result interpretation. Metaproteomic data indicate that the urinary pellet obtained from this human subject contain proteins derived from three different bacterial species, each of which is known to be able to cause urinary tract infections (Escherichia coli, Klebsiella pneumonia, Enterobacter hormachei), s in Table 4. The bacterial proteins are highly prevalent indicative of substantial bacterial colonization. The relative abundance of proteins associated with neutrophils and neutrophil degranulation (NIPs) are more balanced with those proteins generally associated with the urothelium (USPs) indicate that there is asymptomatic bacteriuria, with a potentially emerging urinary tract infection. The clinical conclusion would be to monitor the patient to assess if antibiotic treatment in the near future is required.

The results of the metaproteomic analysis of human subject 4 is provided in Table 6.

TABLE 6

| Accession Number | Score | Mass | PSMs | Description | Notes |
|---|---|---|---|---|---|
| UniRef90_P13646 | 9221 | 49900 | 167 (104) | Keratin, type I cytoskeletal 13 n = 13 Tax = Simiiformes | |
| | | | | RepID = K1C13_HUMAN | |
| UniRef90_P04264 | 5194 | 66170 | 121 (74) | Keratin, type II cytoskeletal 1 n = 7 Tax = Eutheria | |
| | | | | RepID = K2C1_HUMAN | |
| UniRef90_E9PFJ3 | 3758 | 65573 | 113 (53) | Uncharacterized protein n = 3 Tax = Simiiformes | USP* |
| | | | | RepID = E9PFJ3_HUMAN | |
| UniRef90_P04083 | 3735 | 38918 | 70 (35) | Annexin A1 n = 14 Tax = Eutheria RepID = ANXA1_HUMAN | USP |
| UniRef90_P06702 | 3047 | 13291 | 52 (27) | Protein S100-A9 n = 5 Tax = Hominoidea | NIP |
| | | | | RepID = S10A9_HUMAN | |
| UniRef90_P07355 | 3018 | 38808 | 44 (30) | Annexin A2 n = 28 Tax = Eutheria RepID = ANXA2_HUMAN | USP |
| UniRef90_O60437 | 1468 | 205193 | 27 (17) | Periplakin n = 3 Tax = Hominoidea RepID = PEPL_HUMAN | |
| UniRef90_A8K2U0 | 1152 | 162430 | 19 (14) | Alpha-2-macroglobulin-like protein 1 n = 17 | |
| | | | | Tax = Simiiformes RepID = A2ML1_HUMAN | |
| UniRef90_B4DR52 | 1150 | 18087 | 23 (13) | Histone H2B n = 5 Tax = Euarchontoglires | |
| | | | | RepID = B4DR52_HUMAN | |
| UniRef90_P29508 | 1120 | 44594 | 27 (12) | Serpin B3 n = 14 Tax = Hominoidea RepID = SPB3_HUMAN | USP |
| UniRef90_P04792 | 1108 | 22826 | 18 (10) | Heat shock protein beta-1 n = 6 Tax = Simiiformes | USP |
| | | | | RepID = HSPB1_HUMAN | |
| UniRef90_Q6N094 | 1079 | 53264 | 23 (14) | Putative uncharacterized protein DKFZp686O01196 n = 3 | |
| | | | | Tax = Homo sapiens RepID = Q6N094_HUMAN | |
| UniRef90_P31947 | 968 | 27871 | 20 (10) | 14-3-3 protein sigma n = 20 Tax = Eutheria | |
| | | | | RepID = 1433S_HUMAN | |
| UniRef90_P63261 | 946 | 42108 | 21 (8) | Actin, cytoplasmic 2 n = 1334 RepID = ACTG_HUMAN | |
| UniRef90_P15144 | 807 | 109870 | 12 (8) | Aminopeptidase N n = 10 Tax = Catarrhini | |
| | | | | RepID = AMPN_HUMAN | |
| UniRef90_P05109 | 799 | 10885 | 50 (14) | Protein S100-A8 n = 5 Tax = Hominoidea | NIP |
| | | | | RepID = S10A8_HUMAN | |
| UniRef90_P04080 | 745 | 11190 | 15 (6) | Cystatin-B n = 14 Tax = Simiiformes RepID = CYTB_HUMAN | USP |
| UniRef90_P62937 | 742 | 18229 | 15 (8) | Peptidyl-prolyl cis-trans isomerase A n = 98 Tax = Theria | USP |
| | | | | RepID = PPIA_HUMAN | |
| UniRef90_P09211 | 691 | 23569 | 11 (7) | Glutathione S-transferase P n = 4 Tax = Simiiformes | USP |
| | | | | RepID = GSTP1_HUMAN | |
| UniRef90_O43707 | 678 | 105245 | 15 (8) | Alpha-actinin-4 n = 40 Tax = Tetrapoda | |
| | | | | RepID = ACTN4_HUMAN | |
| UniRef90_P31949 | 615 | 11847 | 8 (6) | Protein S100-A11 n = 16 Tax = Simiiformes | USP |
| | | | | RepID = S10AB_HUMAN | |
| UniRef90_P68871 | 606 | 16102 | 8 (5) | Hemoglobin subunit beta n = 102 Tax = Primates | |
| | | | | RepID = HBB_HUMAN | |
| gi\|297205850 | 596 | 43637 | 15 (7) | elongation factor EF1A [Lactobacillus jensenii JV-V16] | BACT |
| UniRef90_Q9UBC9 | 587 | 18598 | 48 (10) | Small proline-rich protein 3 n = 4 Tax = Homininae | |
| | | | | RepID = SPRR3_HUMAN | |
| UniRef90_P55072 | 572 | 89950 | 7 (4) | Transitional endoplasmic reticulum ATPase n = 48 | |
| | | | | Tax = Euteleostomi RepID = TERA_HUMAN | |
| UniRef90_P27482 | 572 | 16937 | 5 (4) | Calmodulin-like protein 3 n = 5 Tax = Euarchontoglires | |
| | | | | RepID = CALL3_HUMAN | |
| UniRef90_UPI000011049E | 543 | 24407 | 13 (8) | IGG CTM01 FAB (LIGHT CHAIN) n = 2 Tax = Homo sapiens | |
| | | | | RepID = UPI000011049E | |
| UniRef90_Q01469 | 510 | 15497 | 11 (4) | Fatty acid-binding protein, epidermal n = 10 | USP |
| | | | | Tax = Simiiformes RepID = FABP5_HUMAN | |
| gi\|297205326 | 507 | 37064 | 6 (6) | D-lactate dehydrogenase [Lactobacillus jensenii JV-V16] | BACT |
| UniRef90_Q13835 | 489 | 84119 | 17 (4) | Plakophilin-1 n = 8 Tax = Eutheria RepID = PKP1_HUMAN | |
| UniRef90_Q4LE79 | 472 | 267367 | 24 (5) | DSP variant protein (Fragment) n = 7 Tax = Eutheria | |
| | | | | RepID = Q4LE79_HUMAN | |
| UniRef90_P47929 | 426 | 15123 | 4 (3) | Galectin 7 n = 7 Tax = Simiiformes RepID = LEG7_HUMAN | |
| UniRef90_P80188 | 426 | 22745 | 6 (3) | Neutrophil gelatinase-associated lipocalin n = 7 | NIP |
| | | | | Tax = Hominidae RepID = NGAL_HUMAN | |
| UniRef90_E9PDK5 | 411 | 46212 | 18 (7) | Annexin A11 n = 3 Tax = Simiiform. RepID = E9PDK5_HUMAN | |
| UniRef90_P07237 | 393 | 57480 | 12 (3) | Protein disulfide-isomerase n = 13 Tax = Eutheria | |
| | | | | RepID = PDIA1_HUMAN | |
| UniRef90_P62158 | 383 | 16827 | 4 (3) | Calmodulin n = 237 Tax = Eukaryota RepID = CALM_HUMAN | |
| UniRef90_P06733 | 370 | 47481 | 8 (4) | Alpha-enolase n = 53 Tax = Euteleostomi | |
| | | | | RepID = ENOA_HUMAN | |
| UniRef90_P37802 | 364 | 22548 | 7 (4) | Transgelin-2 n = 14 Tax = Theria RepID = TAGL2_HUMAN | |
| UniRef90_Q6ZVX7 | 350 | 30942 | 3 (3) | Non-specific cytotoxic cell receptor protein 1 homolog | |
| | | | | n = 5 Tax = Catarrhini RepID = NCRP1_HUMAN | |
| UniRef90_Q9HCY8 | 329 | 11826 | 7 (5) | Protein S100-A14 n = 15 Tax = Eutheria | |
| | | | | RepID = S10AE_HUMAN | |
| UniRef90_P30041 | 326 | 25133 | 8 (3) | Peroxiredoxin-6 n = 17 Tax = Eutheria | |
| | | | | RepID = PRDX6_HUMAN | |
| UniRef90_O96009 | 318 | 45700 | 9 (3) | Napsin-A n = 7 Tax = Simiiformes RepID = NAPSA_HUMAN | |
| UniRef90_B4DQ53 | 318 | 15688 | 9 (3) | Uncharacterized protein n = 1 Tax = Homo sapiens | |
| | | | | RepID = B4DQ53_HUMAN | |
| UniRef90_P15311 | 314 | 69484 | 9 (4) | Ezrin n = 39 Tax = Amniota RepID = EZRI_HUMAN | |
| UniRef90_E9PH67 | 311 | 16728 | 8 (3) | Uncharacterized protein n = 3 Tax = Homo sapiens | |
| | | | | RepID = E9PH67_HUMAN | |
| UniRef90_P02768 | 308 | 71317 | 10 (5) | Serum albumin n = 19 Tax = Catarrhini RepID = ALBU_HUMAN | |
| UniRef90_P0C0S8 | 308 | 14083 | 7 (3) | Histone H2A type 1 n = 295 Tax = Eukaryota | |
| | | | | RepID = H2A1_HUMAN | |
| UniRef90_D6RFL4 | 303 | 23640 | 3 (3) | Uncharacterized protein n = 1 Tax = Homo sapiens | |
| | | | | RepID = D6RFL4_HUMAN | |
| UniRef90_A8MXQ4 | 301 | 30608 | 7 (3) | L-lactate dehydrogenase n = 8 Tax = Simiiformes | |
| | | | | RepID = A8MXQ4_HUMAN | |
| gi\|300362639 | 293 | 37696 | 3 (2) | D-lactate dehydrogenase [Lactobacillus gasseri JV-V03] | BACT |
| UniRef90_B4E1U2 | 293 | 69610 | 4 (2) | Uncharacterized protein n = 2 Tax = Simiiformes | |
| | | | | RepID = B4E1U2_HUMAN | |
| UniRef90_B4DSE2 | 293 | 41992 | 3 (2) | Uncharacterized protein n = 2 Tax = Simiiformes | |
| | | | | RepID = B4DSE2_HUMAN | |
| UniRef90_E7ENQ5 | 293 | 30901 | 2 (2) | Uncharacterized protein n = 2 Tax = Hominoidea | |
| | | | | RepID = E7ENQ5_HUMAN | |
| UniRef90_P16444 | 293 | 46101 | 2 (2) | Dipeptidase 1 n = 4 Tax = Catarrhini RepID = DPEP1_HUMAN | |
| UniRef90_O60235 | 288 | 46748 | 4 (2) | Transmembrane protease serine 11D n = 9 Tax = Catarrhini | |
| | | | | RepID = TM11D_HUMAN | |
| UniRef90_P18054 | 287 | 76615 | 6 (4) | Arachidonate 12-lipoxygenase, 12S-type n = 5 Tax = Eutheria | |
| | | | | RepID = LOX12_HUMAN | |
| UniRef90_Q5RHS7 | 284 | 16027 | 3 (2) | S100 calcium binding protein A2 n = 2 Tax = Catarrhini | |
| | | | | RepID = Q5RHS7_HUMAN | |
| UniRef90_P14923 | 279 | 82434 | 11 (2) | Junction plakoglobin n = 31 Tax = Theria | |
| | | | | RepID = PLAK_HUMAN | |
| UniRef90_P02511 | 268 | 20146 | 3 (2) | Alpha-crystallin B chain n = 30 Tax = Theria | |
| | | | | RepID = CRYAB_HUMAN | |
| gi\|297205635 | 255 | 33254 | 2 (2) | carbamate kinase [Lactobacillus jensenii JV-V16] | BACT |
| gi\|297205435 | 247 | 51944 | 2 (2) | maltose-6′-phosphate glucosidase [Lactobacillus jensenii | BACT |
| | | | | JV-V16] | |
| UniRef90_F5H0L3 | 243 | 47900 | 8 (3) | 6-phosphogluconate dehydrogenase, decarboxylating n = 5 | |
| | | | | Tax = Eutheria RepID = F5H0L3_HUMAN | |
| gi\|297206011 | 242 | 50167 | 5 (2) | NADH peroxidase [Lactobacillus jensenii JV-V16] | BACT |
| UniRef90_P01042 | 237 | 72996 | 4 (3) | Kininogen-1 n = 11 Tax = Catarrhini RepID = KNG1_HUMAN | USP |
| UniRef90_B7ZLJ4 | 235 | 17611 | 6 (3) | Peroxiredoxin 5 n = 5 Tax = Simiiformes | |
| | | | | RepID = B7ZLJ4_HUMAN | |
| UniRef90_B7Z6Z4 | 230 | 26975 | 4 (2) | Uncharacterized protein n = 1 Tax = Homo sapiens | |
| | | | | RepID = B7Z6Z4_HUMAN | |
| gi\|27468618 | 227 | 51524 | 5 (2) | ATP synthase F0F1 subunit beta [Staphylococcus | BACT |
| | | | | epidermidis ATCC 12228] | |
| gi\|297205883 | 222 | 63279 | 7 (4) | pyruvate kinase [Lactobacillus jensenii JV-V16] | BACT |
| gi\|297205636 | 214 | 46324 | 4 (3) | arginine deiminase [Lactobacillus jensenii JV-V16] | BACT |
| UniRef90_P31151 | 211 | 11578 | 9 (3) | Protein S100-A7 n = 7 Tax = Catarrhini | |
| | | | | RepID = S10A7_HUMAN | |
| UniRef90_Q96FQ6 | 211 | 11851 | 3 (3) | Protein S100-A16 n = 4 Tax = Simiiformes | |
| | | | | RepID = S10AG_HUMAN | |
| UniRef90_E7EUT5 | 209 | 28024 | 6 (2) | Glyceraldehyde-3-phosphate dehydrogenase n = 3 | USP |
| | | | | Tax = Eutheria RepID = E7EUT5_HUMAN | |
| UniRef90_B7ZLH8 | 206 | 235047 | 13 (3) | EVPL protein n = 4 Tax = Simiiformes | |
| | | | | RepID = B7ZLH8_HUMAN | |
| gi\|297205762 | 203 | 49500 | 3 (2) | glucose-6-phosphate isomerase [Lactobacillus jensenii JV- | BACT |
| | | | | V16] | |
| gi\|297206263 | 202 | 43078 | 11 (3) | phosphoglycerate kinase [Lactobacillus jensenii JV-V16] | BACT |
| UniRef90_P05386 | 200 | 11621 | 4 (3) | 60S acidic ribosomal protein P1 n = 40 Tax = Eutheria | |
| | | | | RepID = RLA1_HUMAN | |
| UniRef90_F5H5D3 | 193 | 58606 | 4 (2) | Uncharacterized protein n = 5 Tax = Simiiformes | |
| | | | | RepID = F5H5D3_HUMAN | |
| gi\|300813056 | 191 | 36656 | 6 (2) | glyceraldehyde-3-phosphate dehydrogenase, type I | BACT |
| | | | | [Lactobacillus delbrueckii subsp. bulgaricus PB2003] | |
| UniRef90_B4DUU6 | 187 | 56864 | 7 (3) | Pyruvate kinase n = 4 Tax = Simiiformes | BACT |
| | | | | RepID = B4DUU6_HUMAN | |
| gi\|297205493 | 185 | 59500 | 4 (2) | possible Bilirubin oxidase [Lactobacillus jensenii JV-V16] | BACT |
| UniRef90_P61158 | 184 | 47797 | 3 (2) | Actin-related protein 3 n = 57 Tax = Euteleostomi | |
| | | | | RepID = ARP3_HUMAN | |
| UniRef90_B2MUD5 | 181 | 21048 | 7 (2) | Neutrophil elastase (Fragment) n = 1 Tax = Homo sapiens | NIP |
| | | | | RepID = B2MUD5_HUMAN | |
| UniRef90_P13639 | 179 | 96246 | 7 (2) | Elongation factor 2 n = 65 Tax = Euteleostomi | |
| | | | | RepID = EF2_HUMAN | |
| UniRef90_E7EMM4 | 178 | 42169 | 4 (2) | Uncharacterized protein n = 4 Tax = Simiiformes | |
| | | | | RepID = E7EMM4_HUMAN | |
| UniRef90_O43175 | 177 | 57356 | 3 (2) | D-3-phosphoglycerate dehydrogenase n = 18 Tax = Eutheria | |
| | | | | RepID = SERA_HUMAN | |
| gi\|297206261 | 170 | 47020 | 7 (2) | enolase [Lactobacillus jensenii JV-V16] | BACT |
| UniRef90_P29373 | 162 | 15854 | 3 (2) | Cellular retinoic acid-binding protein 2 n = 21 Tax = Theria | |
| | | | | RepID = RABP2_HUMAN | |
| UniRef90_P02545 | 160 | 74380 | 5 (2) | Prelamin-A/C n = 44 Tax = Eutheria RepID = LMNA_HUMAN | |
| UniRef90_P02760 | 156 | 39886 | 4 (2) | Protein AMBP n = 5 Tax = Simiiformes | |
| | | | | RepID = AMBP_HUMAN | |
| UniRef90_P11142 | 155 | 71082 | 5 (2) | Heat shock cognate 71 kDa protein n = 167 Tax = Metazoa | |
| | | | | RepID = HSP7C_HUMAN | |
| UniRef90_A4D2J6 | 155 | 28544 | 7 (3) | Phosphoglycerate mutase n = 1 Tax = Homo sapiens | |
| | | | | RepID = A4D2J6_HUMAN | |
| gi\|297205634 | 153 | 37327 | 3 (1) | ornithine carbamoyltransferase [Lactobacillus jensenii JV- | BACT |
| | | | | V16] | |
| UniRef90_Q01518 | 153 | 52325 | 2 (1) | Adenylyl cyclase-associated protein 1 n = 38 Tax = Eutheria | |
| | | | | RepID = CAP1_HUMAN | |
| gi\|297205218 | 153 | 41362 | 2 (1) | ABC superfamily ATP binding cassette transporter, ABC | BACT |
| | | | | protein [Lactobacillus jensenii JV-V16] | |
| UniRef90_P0CG04 | 153 | 11512 | 4 (1) | Ig lambda-1 chain C regions n = 8 Tax = Hominidae | |
| | | | | RepID = LAC1_HUMAN | |
| UniRef90_Q86T26 | 153 | 46877 | 6 (1) | Transmembrane protease serine 11B n = 5 Tax = Simiiformes | |
| | | | | RepID = TM11B_HUMAN | |

Legend.
Accession numbers are from the Uniprot or the NCBI Entrez Med databases (see method section).
The scores are derived from the database searches with the Mascot v.2.3 algorithms (Matrix Bioscience). A high score is indicative of more peptide identifications for a given protein combined with higher confidence identifications of the peptides.
Masses are the relative molecular mass values for the entire protein, as annotated in the searched databases.
PSMs (peptide-spectral matches) provide the semi-quantitative abundance value for an identified protein. Only statistically significant peptide-spectral matches for a protein sequence are counted. In parentheses are the rank = 1 peptides. The latter peptide counts (rank = 1) are selected for semi-quantitative analysis.
Description lists the protein name, protein name abbreviation and species (e.g. a bacterial species and human).
Notes are provided for the association of proteins with expression in as well as secretion by neutrophils and inflammation (NIP), abundance in and secretion or shedding by urothelium (USP), and a bacterial pathogen (BACT).
USP* Uromodulin is released into the urine in very high abundance. There is no evidence that this protein is released in higher abundance when bacteria colonize the urogenital tract and inflammation occurs.
Result interpretation. Metaproteomic data indicate that the urinary pellet obtained from this human subject contain proteins derived from two different bacterial species, both of which only rarely cause urinary tract infections (Lactobacillus jensenii, Staphylococcus epidermidis). The bacterial proteins are of low abundance compared to the human host proteins in the urinary pellet. # The relative abundance of proteins associated with neutrophils and other phagocytes (NIPs) is very low com antibiotic treatment.

1. A method comprising the steps of:
(a) preparing a urinary pellet from a urine sample or sample prepared from a uretheral catheter associated biofilm;
(b) preparing a protein mixture from the urinary pellet; and
(c) analyzing the protein mixture using a metaproteomic technology approach.

2. The method of claim 1, wherein step (a) is performed by centrifuging the sample to obtain an insoluble pellet and re-suspending the pellet in a buffered solution.

3. The method of claim 1, wherein step (b) is performed by subjecting the urinary pellet to appropriate conditions for lysing and solubilizing microbial and/or host cells and solubilizing microbial and/or host extracellular aggregates so that the majority of proteins present in the pellet are susceptible to proteolytic digestion.

4. The method of claim 3, wherein the solubilized proteins derived from the urinary pellet are contacted with and fully or partially digested by an endopeptidase.

5. The method of claim 1, wherein the metaproteomic analysis comprises the steps of analyzing the digested protein mixture using an appropriate MS or MS/MS system to generate mass spectral data and searching the MS data with a compilation of protein sequence databases derived from annotated microbial genomes that represent at least some of the microbial species and the colonized mammalian host organism in the mixture.

6. The method of claim 5, wherein the searching step is comprised of a computational comparison of peptide m/z values derived from the proteins in the database via in silico digestion with a given endopeptidase to experimentally observed peptide m/z values.

7. A method for diagnosing a subject with a urogenital tract or kidney infection, comprising the steps of:
(a) preparing a urinary pellet from a urine sample or sample prepared from a uretheral catheter associated biofilm;
(b) preparing a protein mixture from the urinary pellet;
(c) analyzing the protein mixture using an appropriate MS or MS/MS system to generate mass spectral data;
(d) searching the mass spectral data with a compilation of protein sequence databases, which include microbial proteins derived from urinary tract pathogens and associated with urinary tract infections, host response proteins and host non-response proteins; and
(e) identifying and quantifying proteins, wherein identification of microbial proteins associated with urinary tract infections and a relatively higher quantitative level of host response proteins to host non-response proteins indicates that the subject has a urogenital tract or kidney infection.

8. The method of claim 7, wherein the protein identification and quantification steps are performed using a computational algorithm that identifies peptide spectral matches supported by calculations of statistical significance of such matches.

9. The method of claim 7, wherein step (a) is performed by centrifuging the sample to obtain an insoluble pellet and re-suspending the insoluble pellet in a buffered solution.

10. The method of claim 7, wherein step (b) is performed by subjecting the urinary pellet to appropriate conditions for lysing and solubilizing microbial and/or host proteins present in the pellet.

11. The method of claim 10, wherein the solubilized proteins derived from the urinary pellet are contacted with and fully or partially digested by an endopeptidase.

12. The method of claim 7, wherein one or more consecutive liquid chromatography (LC) steps are performed to decrease peptide complexity in the sample prior to mass spectral analysis.

13. The method of claim 7, wherein host response proteins are selected from the group consisting of: antimicrobial, pro-inflammatory, cell adhesion, immune system activating, and pro-apoptotic proteins, proteins highly expressed in macrophages and polymorphonuclear neutrophils and proteins released from neutrophil granules or cytoplasm during degranulation and/or released from neutrophils during extracellular trap formation.

14. The method of claim 7, further comprising the step of performing a 16S rRNA sequencing-based metagenomic analysis of the urinary pellet.

15. The method of claim 14, wherein the 16S rRNA metagenomic analysis serves to direct the selection of protein sequence databases to be searched and/or to confirm the metaproteomic identifications of microbial species.

16. A method for diagnosing asymptomatic bacteriuria in a subject, comprising the steps of:
(a) preparing a urinary pellet from a urine sample or sample prepared from a urethral catheter associated biofilm;
(b) preparing a protein mixture from the urinary pellet;
(c) analyzing the protein mixture using an appropriate MS or MS/MS system to generate mass spectral data; and
(d) searching the mass spectral data with a compilation of protein sequence databases, which include microbial proteins derived from urinary tract colonizing microbes which may include urinary tract pathogens and host response and host non-response proteins,
wherein identification and quantification of microbial proteins derived from urinary tract colonizing microbes which may include urinary tract pathogens and a relatively low quantitative level of host response proteins relative to host non-response proteins indicates that the subject has asymptomatic bacteriuria.

17. The method of claim 16, wherein the identification step is performed using a computational algorithm that identifies peptide spectral matches.

18. The method of claim 17, wherein the searches performed with such an algorithm consist of a computational comparison of peptide m/z values derived from the proteins in the database via in silico digestion with a given endopeptidase to experimentally observed peptide m/z values.

19. The method of claim 16, wherein step (a) is performed by centrifuging the sample to obtain an insoluble pellet and re-suspending the insoluble pellet in a buffered solution.

20. The method of claim 16, wherein step (b) is performed by subjecting the urinary pellet to appropriate conditions for lysing and solubilizing microbial and/or host cells and solubilizing microbial and/or host extracellular aggregates so that the majority of proteins present in the pellet are susceptible to proteolytic digestion.

21. The method of claim 20, wherein the appropriate conditions include subjecting the urinary pellet to a digestion with an endopeptidase.

22. The method of claim 16, wherein one or more consecutive liquid chromatography steps are performed to decrease peptide complexity in the sample prior to mass spectral analysis.

23. The method of claim 16, wherein host non-response proteins are selected from the group consisting of: ubiquitous proteins shed from the urinary and bladder epithelium, anti-inflammatory, anti-apoptotic, cytoskeleton-associated and protease inhibitory proteins.

24. The method of claim 7, where the majority of the identified, host organism-derived proteins are those released from neutrophils during neutrophil degranulation and/or neutrophil extracellular trap formation.

25. The method of claim 7, where microbial proteins selected from the group consisting of:
enzymes associated with reactive oxygen and nitrogen species detoxification, iron acquisition proteins, efflux pumps for antibiotics and xenobiotics are among the highly abundant microbial proteins identified in the presence of host response proteins.
